(19) United States
(12) Patent Application Publication
(10) Pub. No.: US 2025/0178995 A1
(43) Pub. Date: Jun. 5, 2025

(54) COMPOSITIONS AND METHODS FOR THE PRODUCTION OF GLUCARIC ACID

(71) Applicant: Solugen, Inc., Houston, TX (US)

(72) Inventors: Toni M. Lee, Missouri City, TX (US); David Weiner, Houston, TX (US); Konrad V. Miller, Sugar Land (TX); Gaurab Chakrabarti, Houston, TX (US); Sean Hunt, Houston, TX (US)

(73) Assignee: Solugen, Inc., Houston, TX (US)

(21) Appl. No.: 18/965,379

(22) Filed: Dec. 2, 2024

Related U.S. Application Data
(60) Provisional application No. 63/604,831, filed on Nov. 30, 2023.

(51) Int. Cl. C07C 31/26; B01J 31/00

(52) U.S. Cl. CPC C07C 31/26; B01J 31/003

(57) ABSTRACT

A method of preparing glucaric acid comprising contacting D-glucose and oxygen with a first catalyst composition comprising a first copper radical oxidase, a single electron oxidizer, and a small molecule activator under conditions suitable for formation of glucodialdose; and contacting glucodialdose and oxygen with a second catalyst composition comprising a second copper radical oxidase, a single electron oxidizer and a small molecule activator under conditions suitable for the formation of a product mixture comprising glucaric acid or salts thereof. A method comprising contacting a sugar with a catalyst composition comprising an oxidoreductase, a single electron oxidizer and a small molecule activator under conditions suitable for the formation of one or more oxidized sugar oxidation products comprising glucaric acid wherein the oxidoreductase comprises at least two copper radical oxidases, at least two mutated copper radical oxidases or combinations thereof.

### CROSS REFERENCE TO RELATED APPLICATIONS

This application claims priority to U.S. Provisional Patent Application No. 63/604,831 filed on Nov. 30, 3023 and entitled, “COMPOSITIONS AND METHODS FOR THE PRODUCTION OF GLUCARIC ACID,” which is hereby incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML file, created on Nov. 25, 2024, is named “23ENZ008PCT_3416-18001 SEQUENCES11252024.xml” and is 94,208 bytes in size.

### TECHNICAL FIELD

The present disclosure relates generally to the production of high value chemicals. More particularly, the present disclosure relates to the production of glucaric acid from reactants sourced from renewable resources.

### BACKGROUND

Over the last 150 years, synthesis of inexpensive chemicals from fossilized forms of carbon (e.g. oil, coal, natural gas) has dramatically altered society through their broad applications, ranging from cosmetics to plastics. This petroleum-based carbon feedstock generates a small collection of platform chemicals from which highly efficient chemical conversions lead to the manufacture of a large variety of chemical products. However, the current approach to producing these carbon-based chemicals is inherently non-sustainable as feedstocks that required millions of years to form are being depleted. The creation of a truly sustainable chemical industry will only occur when the timescale of the feedstock formation matches the timescale of its utilization to make chemicals.

For example, the principal methodologies for producing glucaric acid commercially involve 1) nitric acid oxidation or 2) transition metal catalyzed oxidation. The drawbacks of these commercial methods include (i) the generation of a significant amount of hazardous nitrogen oxide (NOx) gas and (ii) the processes are highly exothermic leading to controllability issues. The transition metal catalyzed (e.g., Pt or Pd) method for glucaric acid production uses glucaric acid as an intermediate in the production of bio-based adipic acid.

Other methods for the production of glucaric acid include a microbial method of producing high-purity glucaric acid using as catalysts myo-inositol-1-phosphate synthase from S. cerevisiae, mouse myo-inositol oxygenase, and P. syringae uronate dehydrogenase. Microbial methods, however, can suffer from product separation issues leading to high chemical costs, limiting usage as a commodity chemical. An ongoing need exists for novel glucaric acid production methods that lack some of the aforementioned challenges.

### SUMMARY

A method of preparing glucaric acid comprising contacting D-glucose and oxygen with a first catalyst composition comprising a first copper radical oxidase, a single electron oxidizer, and a small molecule activator under conditions suitable for formation of glucodialdose; and contacting glucodialdose and oxygen with a second catalyst composition comprising a second copper radical oxidase, a single electron oxidizer and a small molecule activator under conditions suitable for the formation of a product mixture comprising glucaric acid or salts thereof.

A method comprising contacting a sugar with a catalyst composition comprising an oxidoreductase, a single electron oxidizer and a small molecule activator under conditions suitable for the formation of one or more oxidized sugar oxidation products comprising glucaric acid wherein the oxidoreductase comprises at least two copper radical oxidases, at least two mutated copper radical oxidases or combinations thereof.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF DRAWINGS

For a detailed description of various exemplary embodiments, reference will now be made to the accompanying drawings in which:

FIG. 1 schematically depicts a reaction of the present disclosure for generation of glucaric acid from D-glucose.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing FIGURES are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

In the following discussion and in the claims, the terms “including” and “comprising” are used in an open-ended fashion, and thus should be interpreted to mean “including, but not limited to . . . ” As used herein, the phrases “consist(s) of” and “consisting of” are used to refer to exclusive components of a composition, meaning only those expressly recited components are included in the composition; whereas the phrases “consist(s) essentially of” and “consisting essentially of” are used to refer to the primary components of a composition, meaning that only small or trace amounts of components other than the expressly recited components (e.g., impurities, byproducts, etc.) may be included in the composition. For example, a composition consisting of X and Y refers to a composition that only includes X and Y, and thus, does not include any other components; and a composition consisting essentially of X and Y refers to a composition that primarily comprises X and Y, but may include small or trace amounts of components other than X and Y. In embodiments described herein, any such small or trace amounts of components other than those expressly recited following the phrase “consist(s) essentially of” or “consisting essentially of” preferably represent less than 5.0 wt % of the composition, more preferably less than 4.0 wt % of the composition, even more preferably less than 3.0 wt % of the composition, and still more preferably less than 1.0 wt % of the composition. Use of broader terms such as comprises, includes, having, etc. should be understood to provide support for narrower terms such as consisting of, consisting essentially of, comprised substantially of, etc. Use of the term “optionally” with respect to any element of a claim is intended to mean that the subject element is required, or alternatively, is not required. Both alternatives are intended to be within the scope of the claim.

Disclosed herein are chemoenzymatic methods of producing glucaric acid from a renewable resource such as a sugar. Advantageously, the disclosed chemoenzymatic method of glucaric acid production involves the use of benign conditions such as aqueous solvents and low temperatures. In one or more aspects, the disclosed compositions and methods are combined with processes for the formation of hexamethyldiamine which is subsequently utilized in the production of polyamides such as poly [imino (1,6-dioxohexamethylene) iminohexamethylene] (Nylon 66).

Advantageously, the presently disclosed methods utilize bio-based feedstocks, for example, feedstocks derived from biomass, as reactants resulting in the production of glucaric acid with high selectivity and minimized formation of toxic waste products.

In one or more aspects, a method of the present disclosure comprises contacting one or more sugars and subsequent reaction intermediates with a plurality of copper radical oxidases (CROs) under conditions suitable for the formation of glucaric acid.

In one or more aspects, the sugar comprises glucose. CROs are a class of non-flavoprotein alcohol oxidoreductases that employ molecular oxygen as a terminal electron acceptor to generate hydrogen peroxide. CROs have been labeled ‘green’ small-molecule oxidation catalysts as they lack dependence on an organic cofactor and utilizesmolecular oxygen as a cosubstrate.

Nonlimiting examples of CROs suitable for use in the present disclosure include glyoxal oxidases (EC 1.1.3.-, GLOX) and galactose 6-oxidases (EC 1.1.3.9, GAO). GLOXes typically function on aldehydes such as methylglyoxal to produce acids. One GLOX from Phanerochaete chrysosporium primarily accepts alpha-dicarbonyl and alpha-hydroxycarbonyls. In one or more aspects, the CRO is sourced from Pycnoporus cinnabarinus. Pycnoporus cinnabarinus was found to express three GLOXes, one of which has been found to function on methylglyoxal while the other two show high catalytic efficiency for glyoxylic acid.

In an aspect, the CRO is a GAO, alternatively a mutated GAO. GAOs function by oxidizing six-carbon (C6) or similar alcohols of galactose or other sugars to produce aldehydes. A particular example of a CRO is the GAO from Fusarium graminearum. Two additional CROs capable of oxidizing aliphatic alcohols are CgrAlcOx and CglAlcOx, which are derived from Colletotrichum graminicola and C. gloeosporioides, respectively. Another example is aryl-alcohol oxidase (CgrAAO) from C. graminicola.

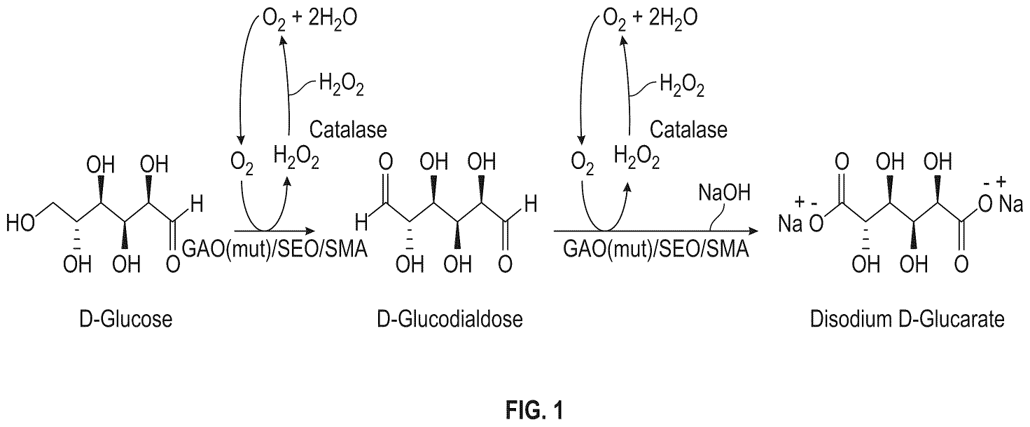

In one or more aspects, a method of the present disclosure is depicted in FIG. 1. With reference to FIG. 1, a sugar (e.g., D-glucose) is reacted with a first CRO (designated CRO1) under conditions suitable for the formation of an intermediate, D-glucodialdose (GDA). In one or more aspects, conditions suitable for the formation of GDA include the presence of a small molecule activator (SMA) and a single electron oxidizer (SEO) such as a peroxidase; both of which facilitate the catalytic activity of the CRO1.

Without wishing to be limited by theory, the SEO performs a single electron oxidation on the SMA to generate the free radical form (SMA^{⋅+}). The SMA^{⋅+} can reverse or return a CRO (e.g., GAO) to the active state. Without wishing to be limited by theory, reversing and/or returning a CRO to the active state may comprise oxidation of one or more metals of the CRO at the active site of the enzyme to an active oxidation state.

In one or more aspects, an SMA suitable for use in this disclosure is a molecule that (i) is capable of stabilizing a free radical, (ii) can serve as a substrate for a SEO in a single electron oxidation reaction, and iii) is capable of restoring the active state of a CRO as evidenced by detectable activity (e.g., formation of product or oxygen consumption).

Nonlimiting examples of SMAs suitable for use in the present disclosure include L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, Allura Red AC, menadione, p-cresol, Fast green FCF, Brilliant Blue FCF, methylisothiazolinone, caffeine, veratryl alcohol, fluorescein, and combinations thereof. The structures of several of these SMAs are depicted in Table 1.

TABLE 1

| | |
|---|---|
| L-tryptophan | 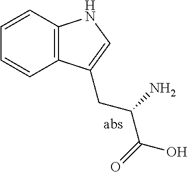 [SMILES: N[C@@H](Cc1c[nH]c2ccccc12)C(=O)O] |
| 2-mercapto- benzothiazole | 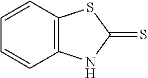 |
| L-histidine | 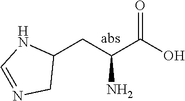 [SMILES: N[C@@H](CC1CN=CN1)C(=O)O] |
| Methylchloro- isothiazolinone | 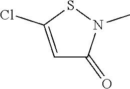 |
| o-dianisidine | 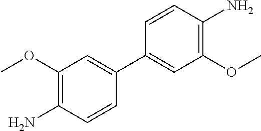 |
| ABTS | 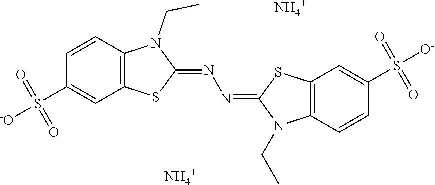 |
| 4-aminoantipyrine | 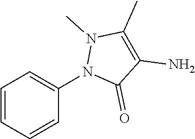 |
| L-tyrosine | 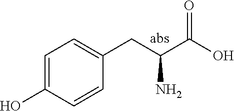 [SMILES: N[C@@H](Cc1ccc(O)cc1)C(=O)O] |
| (2,2,6,6- Tetramethylpiperidin- 1-yl)oxyl | 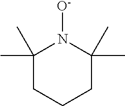 |
| chloromethyl- isothiazolinone | 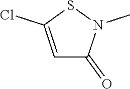 |
| 4-thiazolecarboxylic acid | 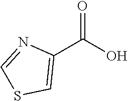 |
| Sunset yellow FCF | 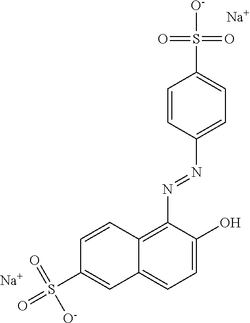 [SMILES: [Na+] ; O=S(=O)([O-])c1ccc(/N=N/c2c(O)ccc3cc(S(=O)(=O)[O-])ccc23)cc1 ; [Na+]] |
| Tartrazine | 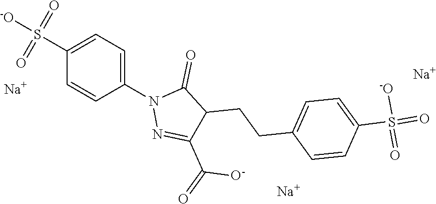 |
| p-benzoquinone | 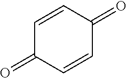 |
| dicoumarol | 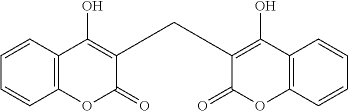 [SMILES: O=c1oc2ccccc2c(O)c1Cc1c(O)c2ccccc2oc1=O] |
| phthalimide | 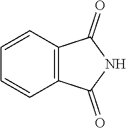 [SMILES: O=C1NC(=O)c2ccccc21] |
| saccharin | 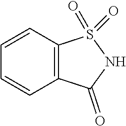 [SMILES: O=C1NS(=O)(=O)c2ccccc21] |
| phthalic anhydride | 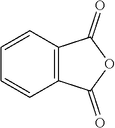 [SMILES: O=C1OC(=O)c2ccccc21] |
| Erythrosine B | 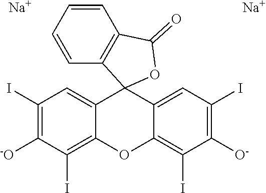 |
| 2-aminobenzothiazole | 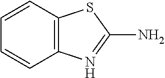 |
| benzothiazole | 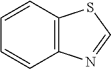 |
| thiabendazole | 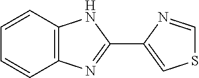 [SMILES: c1ccc2[nH]c(-c3cscn3)nc2c1] |
| 2-Hydroxybenzothiazole | 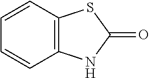 |
| Phenothiazine | 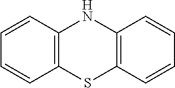 [SMILES: c1ccc2c(c1)Nc1ccccc1S2] |
| 6-aminobenzothiazole | 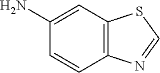 |
| Indigo carmine | 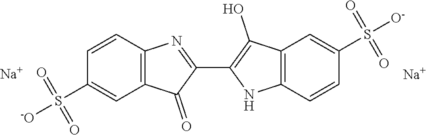 |
| naphthalimide | 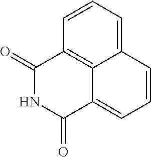 [SMILES: O=C1NC(=O)c2cccc3cccc1c23] |
| 2-aminothiazole | 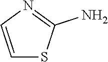 |
| thiazole | 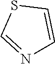 |
| 2H-1,4- Benzothiazin-3(4H)- one | 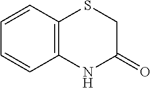 [SMILES: O=C1CSc2ccccc2N1] |
| 2-oxindole | 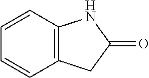 |
| beta-lapachone | 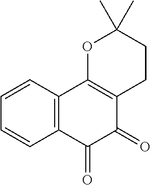 [SMILES: CC1(C)CCC2=C(O1)c1ccccc1C(=O)C2=O] |
| menaquinone | 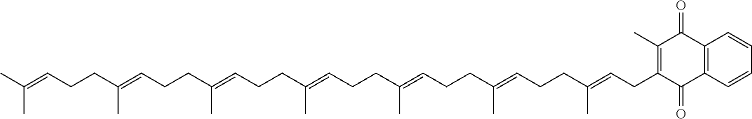 [SMILES: CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O] |
| thiamine | 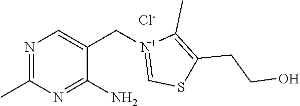 [SMILES: Cc1ncc(C[n+]2csc(CCO)c2C)c(N)n1 ; [Cl-]] |
| 4-Methyl-5- thiazoleethanol | 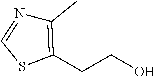 |
| Allura Red AC | 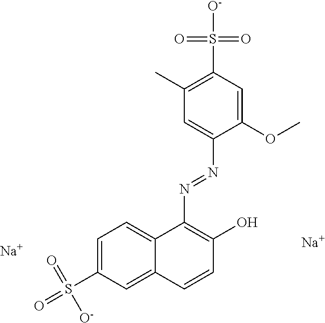 [SMILES: [Na+] ; COc1cc(S(=O)(=O)[O-])c(C)cc1/N=N/c1c(O)ccc2cc(S(=O)(=O)[O-])ccc12 ; [Na+]] |
| Menadione | 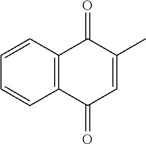 |
| p-cresol | 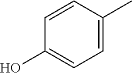 |
| Fast green FCF | 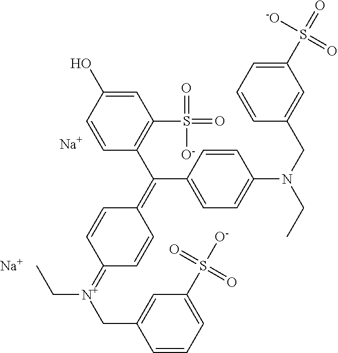 |
| Brilliant Blue FCF | 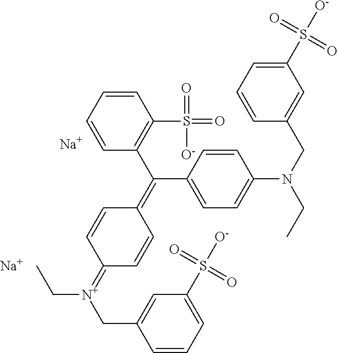 [SMILES: [Na+] ; CCN(Cc1cccc(S(=O)(=O)[O-])c1)c1ccc(C(=C2C=CC(=[N+](CC)Cc3cccc(S(=O)(=O)[O-])c3)C=C2)c2ccccc2S(=O)(=O)[O-])cc1 ; [Na+]] |
| Methylisothiazolinone | 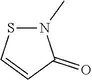 |
| caffeine | 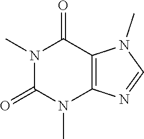 |
| veratryl alcohol | 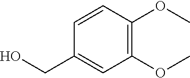 |
| fluorescein | 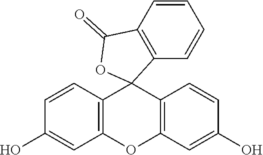 [SMILES: O=C1OC2(c3ccc(O)cc3Oc3cc(O)ccc32)c2ccccc21] |

A SEO suitable for use in the present disclosure can be an enzyme or chemical compound (e.g., peroxidase or ferricyanide, catalase, and the like). The SEO can be any molecule that can elicit the radical state of the SMA by performing a single electron oxidation. In one or more aspects, the SEO is an enzyme. For example, the SEO is an enzyme that can use H₂O₂ as the oxidant which is advantageous as H₂O₂ is produced as a coproduct by a CRO.

In one or more aspects, the SEO is an enzyme such as a laccase, horseradish peroxidase, Dyp-type peroxidase, lactoperoxidase, chloroperoxidase, manganese peroxidase 1, ascorbate peroxidase, dye-decolorizing peroxidase, unspecific peroxygenase, dehaloperoxidase, catalase-peroxidase, lignin peroxidase, soybean seed coat peroxidase, isoforms thereof or combinations thereof.

In one or more aspects, a catalytic composition comprises a GAO, an SEO and a SMA. The catalytic composition may comprise an GAO in an amount ranging from about 0.01 g/L to about 1 g/L, additionally or alternatively, from about 0.1 g/L to about 1 g/L, additionally or alternatively, from about 0.2 g/L to about 1 g/L, additionally or alternatively, from about 0.4 g/L to about 1 g/L, additionally or alternatively, from about 0.6 g/L to about 1 g/L, additionally or alternatively, from about 0.75 g/L to 1 g/L, additionally or alternatively, about 0.01 g/L, about 0.05 g/L, about 0.1 g/L, about 0.2 g/L, about 0.3 g/L, about 0.4 g/L, about 0.5 g/L, about 0.6 g/L, about 0.7 g/L, about 0.8 g/L, about 0.9 g/L or, additionally or alternatively, about 1 g/L. The catalytic composition may comprise an SEO in an amount ranging from about 1 mg/L to about 250 mg/L, additionally or alternatively, from about 5 mg/L to about 250 mg/L, additionally or alternatively, from about 10 mg/L to about 250 mg/L, additionally or alternatively, from about 25 mg/L to about 250 mg/L, additionally or alternatively, from about 50 mg/L to find 250 mg/L, additionally or alternatively, from about 75 mg/L to 250 mg/L, additionally or alternatively, from about 100 mg/L to 250 mg/L, additionally or alternatively, from about 150 mg/L to 250 mg/L, additionally or alternatively, about 1 mg/L, about 5 mg/L, about 10 mg/L, about 15 mg/L, about 20 mg/L, about 25 mg/L, about 30 mg/L, about 35 mg/L, about 40 mg/L, about 45 mg/L, about 50 mg/L, about 55 mg/L, about 60 mg/L, about 65 mg/L, about 70 mg/L, about 75 mg/L, about 80 mg/L, about 85 mg/L, about 90 mg/L, about 95 mg/L, about 100 mg/L, about 105 mg/L, about 110 mg/L, about 115 mg/L, about 120 mg/L, about 125 mg/L, about 130 mg/L, about 135 mg/L, about 140 mg/L, about 145 mg/L, about 150 mg/L, about 155 mg/L, about 160 mg/L, about 165 mg/L, about 170 mg/L, about 175 mg/L, about 180 mg/L, about 185 mg/L, about 190 mg/L, about 195 mg/L, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L or, additionally or alternatively, about 250 mg/L. The catalytic composition may comprise an SMA in an amount ranging from about 1 ppm to about 500 ppm, additionally or alternatively, from about 5 ppm to about 500 ppm, additionally or alternatively, from about 10 ppm to about 500 ppm, additionally or alternatively, from about 20 ppm to about 500 ppm, additionally or alternatively, or from about 40 ppm to about 400 ppm, additionally or alternatively, from about 50 ppm to about 350 ppm additionally or alternatively, from about 75 ppm to about 200 ppm additionally or alternatively, about 1 ppm, about 5 ppm, about 10 ppm, about 15 ppm, about 20 ppm, about 25 ppm, about 30 ppm, about 35 ppm, about 40 ppm, about 45 ppm, about 50 ppm, about 55 ppm, about 60 ppm, about 65 ppm, about 70 ppm, about 75 ppm, about 80 ppm, about 85 ppm, about 90 ppm, about 95 ppm, about 100 ppm, about 105 ppm, about 110 ppm, about 115 ppm, about 120 ppm, about 125 ppm, about 130 ppm, about 135 ppm, about 140 ppm, about 145 ppm, about 150 ppm, about 155 ppm, about 160 ppm, about 165 ppm, about 170 ppm, about 175 ppm, about 180 ppm, about 185 ppm, about 190 ppm, about 195 ppm, about 200 ppm, about 205 ppm, about 210 ppm, about 215 ppm, about 220 ppm, about 225 ppm, about 230 ppm, about 235 ppm, about 240 ppm, about 245 ppm, about 250 ppm, about 255 ppm, about 260 ppm, about 265 ppm, about 270 ppm, about 275 ppm, about 280 ppm, about 285 ppm, about 290 ppm, about 295 ppm, about 300 ppm, about 305 ppm, about 310 ppm, about 315 ppm, about 320 ppm, about 325 ppm, about 330 ppm, about 335 ppm, about 340 ppm, about 345 ppm, about 350 ppm, about 355 ppm, about 360 ppm, about 365 ppm, about 370 ppm, about 375 ppm, about 380 ppm, about 385 ppm, about 390 ppm, about 395 ppm, about 400 ppm, about 405 ppm, about 410 ppm, about 415 ppm, about 420 ppm, about 425 ppm, about 430 ppm, about 435 ppm, about 440 ppm, about 445 ppm, about 450 ppm, about 455 ppm, about 460 ppm, about 465 ppm, about 470 ppm, about 475 ppm, about 480 ppm, about 485 ppm, about 490 ppm, about 495 ppm, or, additionally or alternatively, about 500 ppm.

Conditions suitable for the formation of GDA may include one or more of the following reaction parameters: an amount of sugar (e.g., glucose) of from about 0.1 weight per volume percent (w/v %) to about 60 w/v %, alternatively from about 5 w/v % to about 50 w/v % or alternatively from about 10 w/v % to about 40 w/v %; a temperature ranging from about 1° C. to about 70° C., alternatively from about 5 to about 30° C. or alternatively from about 10° C. to about 25° C.; a suitable buffered media providing a pH ranging from about 5 to about 10, alternatively from about 5.5 to about 9 and a substrate amount ranging from about 6.5 to about 8.5, alternatively from about 6.5 to about 8.5 or alternatively from about 7 to about 8; an oxygen pressure of equal to or less than about 500 psi, alternatively from about 50 psi to about 250 psi or alternatively from about 70 psi to about 150 psi and a reaction time ranging from about 1 hour to about 24 hours or from about 2 hours to about 12 hours or from about 3 hours to about 6 hours.

GDA may be formed at yields that can range from about 50% to about 99%, additionally or alternatively, from about 50% to about 90%, additionally or alternatively, from about 50% to about 80%, additionally or alternatively, at least about 55%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, at least about or at least about 99%. The GDA intermediate formed may be used without further processing in the methods of the present disclosure. In alternative aspects, the GDA intermediate may be subjected to additional processing before (e.g., purification) prior to being utilized in other aspects of the presently disclosed methods. GDA also known as glucodialdose, L-gluco-hexodialdose, and D-gluco-hexodialdose, is a chiral intermediate with multiple industrial applications. The molecular structure corresponds with glucose, which inherently carries an aldehyde at the C1 position, oxidized to the dialdehyde at the C6 position.

In one or more aspects, a method of the present disclosure further comprises contacting the GDA formed with a second CRO (designated CRO2) under conditions suitable for the formation of glucaric acid. In one or more aspects, conditions suitable for the formation of glucaric acid include the presence of an SEO and/or SMA which may be the same as that used to facilitate the catalytic activity of CRO1. Alternatively, the SEO and/or SMA differ from those used to facilitate the catalytic activity of CRO1. In some aspects, CRO1 and CRO2 are the same enzyme, for example a GAO mutated to accept glucose as a substrate. In the alternative, CRO1 and CRO2 are different. For example, CRO 1 may be a GAO mutated to accept glucose as a substrate while CRO2 may be a GLOX mutated to oxidize GDA at the C1 and C6 positions to glucarate.

In one or more aspects, GDA is contacted with a catalytic composition comprising a GLOX, an SEO and a SMA. The catalytic composition may comprise a GLOX in an amount ranging from about 0.01 g/L to about 1 g/L, additionally or alternatively, from about 0.1 g/L to about 1 g/L, additionally or alternatively, from about 0.2 g/L to about 1 g/L, additionally or alternatively, from about 0.4 g/L to about 1 g/L, additionally or alternatively, from about 0.6 g/L to about 1 g/L, additionally or alternatively, from about 0.75 g/L to 1 g/L, additionally or alternatively, about 0.01 g/L, about 0.05 g/L, about 0.1 g/L, about 0.2 g/L, about 0.3 g/L, about 0.4 g/L, about 0.5 g/L, about 0.6 g/L, about 0.7 g/L, about 0.8 g/L, about 0.9 g/L or, additionally or alternatively, about 1 g/L. The catalytic composition may comprise an SEO in an amount ranging from about 1 mg/L to about 250 mg/L, additionally or alternatively, from about 5 mg/L to about 250 mg/L, additionally or alternatively, from about 10 mg/L to about 250 mg/L, additionally or alternatively, from about 25 mg/L to about 250 mg/L, additionally or alternatively, from about 50 mg/L to find 250 mg/L, additionally or alternatively, from about 75 mg/L to 250 mg/L, additionally or alternatively, from about 100 mg/L to 250 mg/L, additionally or alternatively, from about 150 mg/L to 250 mg/L, additionally or alternatively, about 1 mg/L, about 5 mg/L, about 10 mg/L, about 15 mg/L, about 20 mg/L, about 25 mg/L, about 30 mg/L, about 35 mg/L, about 40 mg/L, about 45 mg/L, about 50 mg/L, about 55 mg/L, about 60 mg/L, about 65 mg/L, about 70 mg/L, about 75 mg/L, about 80 mg/L, about 85 mg/L, about 90 mg/L, about 95 mg/L, about 100 mg/L, about 105 mg/L, about 110 mg/L, about 115 mg/L, about 120 mg/L, about 125 mg/L, about 130 mg/L, about 135 mg/L, about 140 mg/L, about 145 mg/L, about 150 mg/L, about 155 mg/L, about 160 mg/L, about 165 mg/L, about 170 mg/L, about 175 mg/L, about 180 mg/L, about 185 mg/L, about 190 mg/L, about 195 mg/L, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L or, additionally or alternatively, about 250 mg/L. The catalytic composition may comprise an SMA in an amount ranging from about 1 ppm to about 500 ppm, additionally or alternatively, from about 5 ppm to about 500 ppm, additionally or alternatively, from about 10 ppm to about 500 ppm, additionally or alternatively, from about 20 ppm to about 500 ppm, additionally or alternatively, or from about 40 ppm to about 400 ppm, additionally or alternatively, from about 50 ppm to about 350 ppm additionally or alternatively, from about 75 ppm to about 200 ppm additionally or alternatively, about 1 ppm, about 5 ppm, about 10 ppm, about 15 ppm, about 20 ppm, about 25 ppm, about 30 ppm, about 35 ppm, about 40 ppm, about 45 ppm, about 50 ppm, about 55 ppm, about 60 ppm, about 65 ppm, about 70 ppm, about 75 ppm, about 80 ppm, about 85 ppm, about 90 ppm, about 95 ppm, about 100 ppm, about 105 ppm, about 110 ppm, about 115 ppm, about 120 ppm, about 125 ppm, about 130 ppm, about 135 ppm, about 140 ppm, about 145 ppm, about 150 ppm, about 155 ppm, about 160 ppm, about 165 ppm, about 170 ppm, about 175 ppm, about 180 ppm, about 185 ppm, about 190 ppm, about 195 ppm, about 200 ppm, about 205 ppm, about 210 ppm, about 215 ppm, about 220 ppm, about 225 ppm, about 230 ppm, about 235 ppm, about 240 ppm, about 245 ppm, about 250 ppm, about 255 ppm, about 260 ppm, about 265 ppm, about 270 ppm, about 275 ppm, about 280 ppm, about 285 ppm, about 290 ppm, about 295 ppm, about 300 ppm, about 305 ppm, about 310 ppm, about 315 ppm, about 320 ppm, about 325 ppm, about 330 ppm, about 335 ppm, about 340 ppm, about 345 ppm, about 350 ppm, about 355 ppm, about 360 ppm, about 365 ppm, about 370 ppm, about 375 ppm, about 380 ppm, about 385 ppm, about 390 ppm, about 395 ppm, about 400 ppm, about 405 ppm, about 410 ppm, about 415 ppm, about 420 ppm, about 425 ppm, about 430 ppm, about 435 ppm, about 440 ppm, about 445 ppm, about 450 ppm, about 455 ppm, about 460 ppm, about 465 ppm, about 470 ppm, about 475 ppm, about 480 ppm, about 485 ppm, about 490 ppm, about 495 ppm, or, additionally or alternatively, about 500 ppm.

In one or more aspects, the reaction disclosed herein is carried out under mild conditions. Conditions suitable for the formation of glucaric acid may include one or more of the following reaction parameters: an amount of GDA of from about 0.1 weight per volume percent (w/v %) to about 60 w/v %, alternatively from about 5 w/v % to about 50 w/v % or alternatively from about 10 w/v % to about 40 w/v %; a temperature ranging from about 5° C. to about 100° C., or from about 10° C. to about 75° C., or from about 15° C. to about 70° C., or from about 25° C. to about 50° C.; a suitable buffered media providing a pH ranging from about 4 to about 9, or from about 5.5 to about 9, or from about 6 to about 8; an oxygen pressure of equal to or less than about 500 psi, alternatively from about 50 psi to about 250 psi or alternatively from about 70 psi to about 150 psi and a reaction time ranging from about 1 hour to about 24 hours or from about 2 hours to about 12 hours or from about 3 hours to about 6 hours.

Glucaric acid may be formed at yields that can range from about 50% to about 99%, additionally or alternatively, from about 50% to about 90%, additionally or alternatively, from about 50% to about 80%, additionally or alternatively, at least about 55%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, at least about or at least about 99%.

In some aspects, the reaction for formation of glucaric acid further includes a caustic agent such as sodium hydroxide or another base which functions to both control pH and as a counterion for produced glucaric acid or glucarate salts.

In some aspects, the catalyst compositions disclosed herein further comprise a catalase in order to decompose the hydrogen peroxide produced during the reaction of the CRO. A catalase is used to break down two molecules of hydrogen peroxide (H₂O₂) into one molecule of oxygen (O₂) and two molecules of water (H₂O). In some aspects, oxygen generated by catalase may be captured and used in the oxidation of additional substrate (e.g., glucose or GDA).

In one or more aspects, the methods of the present disclosure produce high purity glucaric acid or salts thereof, for example, glucaric acid may have a purity of greater than about 70%, or greater than about 90% or greater than about 95%, or from about 70% to about 99%, or from about 72% to about 99%, or from about 75% to about 99%, or from about 78% to about 99%, or from about 80% to about 99%, or from about 82% to about 99%, or from about 84% to about 99%, or from about 86% to about 99%, or from about 88% to about 99%, or from about 90% to about 99%, or from about 92% to about 99%, or from about 94% to about 99%, or from about 96% to about 99%. In an aspect, the product comprises disodium glucarate, monosodium glucarate, potassium glucarate or combinations thereof in crystalline form. In an alternative aspect, the product of the present disclosure comprises a mixture of free acid in combination with salts of glucaric acid (e.g., potassium and/or sodium salt).

In one or more aspects, CRO1 which catalyzes the formation of glucodialdose can be any CRO or mutant thereof capable of oxidizing the C6 of glucose. In one or more aspects, CRO2 which catalyzes the formation of glucaric acid can be any CRO capable of oxidizing the C1 and C6 aldehydes of glucodialdose. In one or more aspects, a GLOX engineered to oxidize the C1 and C6 of GDA could catalyze this reaction.

In an aspect, an enzyme of the type disclosed herein is a wild type enzyme, a functional fragment thereof, or a functional variant thereof. “Fragment” as used herein is meant to include any amino acid sequence shorter than the full-length enzyme, but where the fragment maintains a catalytic activity sufficient to meet some user or process goal. Fragments may include a single contiguous sequence identical to a portion of the biocatalyst sequence. Alternatively, the fragment may have or include several different shorter segments where each segment is identical in amino acid sequence to a different portion of the amino acid sequence of the enzyme but linked via amino acids differing in sequence from the enzyme. Herein, a “functional variant” of the enzyme refers to a polypeptide which has at one or more positions of an amino acid insertion, deletion, or substitution, either conservative or non-conservative, and wherein each of these types of changes may occur alone, or in combination with one or more of the others, and/or one or more times in a given sequence but retains catalytic activity.

In the alternative or in combination with the aforementioned mutations, the enzyme may be mutated to improve the catalytic activity. Mutations may be carried out to enhance the protein or a homolog activity, increase the protein stability in the presence of substrates and products (e.g., hydrogen peroxide) and increase protein yield.

Herein, reference has been made to “sources” of enzyme. It is to be understood this refers to the biomolecule as expressed by the named organism. It is contemplated the enzyme may be obtained from the organism or a version of said enzyme (wildtype or recombinant) and provided as a suitable construct to an appropriate expression system.

In an aspect, any enzyme of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of an enzyme disclosed herein is provided as a recombinant sequence in a vector where the sequence is operatively linked to one or more control or regulatory sequences. “Operatively linked” expression control sequences refer to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and are used herein refer to polynucleotide sequences which affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

In one or more aspects, a CRO suitable for use in the present disclosure is a CRO comprising any of SEQ ID Nos. 1 through 39 or alternatively from about 85% to about 100% sequence identity with any of SEQ ID Nos. 1 through 39. In one or more aspects, a catalase suitable for use in the present disclosure is a catalase defined by of any of SEQ ID Nos. 40 through 44. In one or more aspects, a peroxidase suitable for use in the present disclosure is a peroxidase defined by of any of SEQ ID Nos. 45 through 61 In one or more aspects, a CRO suitable for use in the present disclosure is a CRO defined by of any of SEQ ID Nos.62 through or alternatively from about 85% to about 100% sequence identity with any of SEQ ID Nos. 62 through 65.

In one or more aspects, a glucaric acid product of the type disclosed herein may be used in the production of into adipic acid and subsequently adipic adipodinitrile, which can then be hydrogenated to produce hexamethyldiamine.

### ADDITIONAL DISCLOSURE

A first aspect which is a method of preparing glucaric acid comprising contacting D-glucose and oxygen with a first catalyst composition comprising a first copper radical oxidase, a single electron oxidizer, and a small molecule activator under conditions suitable for formation of glucodialdose; and contacting glucodialdose and oxygen with a second catalyst composition comprising a second copper radical oxidase, a single electron oxidizer and a small molecule activator under conditions suitable for the formation of a product mixture comprising glucaric acid or salts thereof.

A second aspect which is the method of the first aspect wherein the first copper radical oxidase comprises a galactose oxidase, a galactose oxidase mutant or combinations thereof.

A third aspect which is the method of any of the first through second aspects wherein the first copper radical oxidase comprises any of SEQ ID Nos. 1 through 39.

A fourth aspect which is the method of any of the first through third aspects wherein the first copper radical oxidase has from about 85% to about 100% sequence identity with any of SEQ ID Nos. 1 through 39.

A fifth aspect which is the method of any of the first through fourth aspects wherein the second copper radical oxidase comprises a glyoxal oxidase, a glyoxal oxidase mutant or combinations thereof.

A sixth aspect which is the method of any of the first through fifth aspects wherein the second copper radical oxidase comprises any of SEQ ID Nos. 62 through 65.

A seventh aspect which is the method of any of the first through sixth aspects wherein the second copper radical oxidase has from about 85% to about 100% sequence identity with any of SEQ ID Nos. 62 through 65.

An eighth aspect which is the method of any of the first through seventh aspects wherein the small molecule activator comprises L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, Allura Red AC, menadione, p-cresol, Fast green FCF, Brilliant Blue FCF, methylisothiazolinone, caffeine, veratryl alcohol, fluorescein, or combinations thereof.

A ninth aspect which is the method of any of the first through eighth aspects wherein the small molecule activator is present in an amount ranging from about 1 ppm to about 500 ppm.

A tenth aspect which is the method of any of the first through ninth aspects wherein the single electron oxidizer comprises laccase, horseradish peroxidase, dyp-type peroxidase, lactoperoxidase, chloroperoxidase, manganese peroxidase 1, ascorbate peroxidase, dye-decolorizing peroxidase, unspecific peroxygenase, dehaloperoxidase, catalase-peroxidase, lignin peroxidase, soybean seed coat peroxidase, isoforms thereof or combinations thereof.

An eleventh aspect which is the method of any of the first through tenth aspects wherein conditions suitable for formation of glucodialdose, conditions suitable for formation of a product mixture comprising glucaric acid or both comprise an oxygen pressure of equal to or less than about 500 psi.

A twelfth aspect which is the method of any of the first through eleventh aspects further comprising a catalase.

A thirteenth aspect which is the method of any of the first through twelfth aspects wherein the catalase is defined by any SEQ ID No. 40 through SEQ ID No. 44.

A fourteenth aspect which is the method of any of the first through thirteenth aspects further comprising introducing a caustic to the second catalyst composition, the product mixture or both.

A fifteenth aspect which is the method of any of the first through fourteenth aspects wherein the glucodialdose, glucaric acid are formed at yields ranging from about 50% to about 99%.

A sixteenth aspect which is the method of any of the first through fifteenth aspects wherein the glucaric acid or salts thereof have a purity of greater than about 70%.

A seventeenth aspect which is the method of any of the first through sixteenth aspects wherein the product mixture comprising glucaric acid further comprises on or more sugar oxidation products.

An eighteenth aspect which is the method of the seventeenth aspect wherein the one or more sugar oxidation products comprise aldonic acid, uronic acid, aldaric acid, a gluconic acid oxidation product, a gluconate, gluconic acid, glucuronic acid, glucose oxidation products, galactonic acid, galactaric acid, glutamic acid, a lactone of gluconic acid, a lactone of glucaric acid, a lactone of galactaric acid, a lactone of galactonic acid, glucodialdose, 2-ketoglucose, disaccharides, oxidized disaccharides, n-keto-acids, C2 to C6 diacids, salts thereof or combinations thereof.

A nineteenth aspect which is a method comprising contacting a sugar with a catalyst composition comprising an oxidoreductase, a single electron oxidizer and a small molecule activator under conditions suitable for the formation of one or more oxidized sugar oxidation products comprising glucaric acid wherein the oxidoreductase comprises at least two copper radical oxidases, at least two mutated copper radical oxidases or combinations thereof.

A twentieth aspect which is the method of the nineteenth aspect wherein the at least two copper radical oxidases comprises any of SEQ ID Nos. 1 through 39, any of SEQ ID Nos. 62 through 65, have 85% to about 100% sequence identity with any of SEQ ID Nos. 1 through 39 or have 85% to about 100% sequence identity with any of SEQ ID Nos. 62 through 65.

### EXAMPLES

The aspects having been generally described, the following example is given as particular aspects of the disclosure and to demonstrate the practice and advantages thereof. It is understood that the example is given by way of illustration and is not intended to limit the specification or the claims in any manner.

### Example 1

Generation of GAO Mutant for Producing Glucodialdose from Glucose

A GAO mutant capable of converting glucose to GDA was engineered. Following directed evolution and rational enzyme engineering, the improved GAO mutant exhibits a specific activity of 35 U mg⁻¹ on glucose.

### Directed Evolution

Directed evolution of thirty sites within 10 Å of the catalytic copper was performed on a parent sequence containing the following added mutations: 1) R330, Q406T, W290F to introduce less than 1 U mg⁻¹ activity on glucose to GAO, 2) C383S to lower the KM of the enzyme on galactose, and 3) Y405F and Q406E to enhance activity on a D-N-acetyl glucosamine substrate. Other mutations described in Table I were found to have neutral or deleterious effects on glucodialdose-generating activity. The new combination sequence was designated GAO-Mut1. The sequence of GAO-Mut1 contains a “MGHHHHHHSSGHIEGRHM” N-terminal his-tag and linker for expression and purification in E. coli and is SEQ ID NO:66.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A method of preparing glucaric acid, comprising:
contacting D-glucose and oxygen with a first catalyst composition comprising a first copper radical oxidase, a single electron oxidizer, and a small molecule activator under conditions suitable for formation of glucodialdose; and
contacting glucodialdose and oxygen with a second catalyst composition comprising a second copper radical oxidase, a single electron oxidizer and a small molecule activator under conditions suitable for the formation of a product mixture comprising glucaric acid or salts thereof.

2. The method of claim 1, wherein the first copper radical oxidase comprises a galactose oxidase, a galactose oxidase mutant or combinations thereof.

3. The method of claim 1, wherein the first copper radical oxidase comprises any of SEQ ID Nos. 1 through 39.

4. The method of claim 1, wherein the first copper radical oxidase has from about 85% to about 100% sequence identity with any of SEQ ID Nos. 1 through 39.

5. The method of claim 1, wherein the second copper radical oxidase comprises a glyoxal oxidase, a glyoxal oxidase mutant or combinations thereof.

6. The method of claim 1, wherein the second copper radical oxidase comprises any of SEQ ID Nos. 62 through 65.

7. The method of claim 1, wherein the second copper radical oxidase has from about 85% to about 100% sequence identity with any of SEQ ID Nos. 62 through 65.

8. The method of claim 1, wherein the small molecule activator comprises L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, Allura Red AC, menadione, p-cresol, Fast green FCF, Brilliant Blue FCF, methylisothiazolinone, caffeine, veratryl alcohol, fluorescein, or combinations thereof.

9. The method of claim 1, wherein the small molecule activator is present in an amount ranging from about 1 ppm to about 500 ppm.

10. The method of claim 1, wherein the single electron oxidizer comprises laccase, horseradish peroxidase, dyp-type peroxidase, lactoperoxidase, chloroperoxidase, manganese peroxidase 1, ascorbate peroxidase, dye-decolorizing peroxidase, unspecific peroxygenase, dehaloperoxidase, catalase-peroxidase, lignin peroxidase, soybean seed coat peroxidase, isoforms thereof or combinations thereof.

11. The method of claim 1, wherein conditions suitable for formation of glucodialdose, conditions suitable for formation of a product mixture comprising glucaric acid or both comprise an oxygen pressure of equal to or less than about 500 psi.

12. The method of claim 1, further comprising a catalase.

13. The method of claim 1, wherein the catalase is defined by any SEQ ID No. 40 through SEQ ID No. 44.

14. The method of claim 1, further comprising introducing a caustic to the second catalyst composition, the product mixture or both.

15. The method of claim 1, wherein the glucodialdose, glucaric acid are formed at yields ranging from about 50% to about 99%.

16. The method of claim 1, wherein the glucaric acid or salts thereof have a purity of greater than about 70%.

17. The method of claim 1, wherein the product mixture comprising glucaric acid further comprises on or more sugar oxidation products.

18. The method of claim 17, wherein the one or more sugar oxidation products comprise aldonic acid, uronic acid, aldaric acid, a gluconic acid oxidation product, a gluconate, gluconic acid, glucuronic acid, glucose oxidation products, galactonic acid, galactaric acid, glutamic acid, a lactone of gluconic acid, a lactone of glucaric acid, a lactone of galactaric acid, a lactone of galactonic acid, glucodialdose, 2-ketoglucose, disaccharides, oxidized disaccharides, n-keto-acids, C2 to C6 diacids, salts thereof or combinations thereof.

19. A method comprising:
contacting a sugar with a catalyst composition comprising an oxidoreductase, a single electron oxidizer and a small molecule activator under conditions suitable for the formation of one or more oxidized sugar oxidation products comprising glucaric acid wherein the oxidoreductase comprises at least two copper radical oxidases, at least two mutated copper radical oxidases or combinations thereof.

20. The method of claim 19, wherein the at least two copper radical oxidases comprises any of SEQ ID Nos. 1 through 39, any of SEQ ID Nos. 62 through 65, have 85% to about 100% sequence identity with any of SEQ ID Nos. 1 through 39 or have 85% to about 100% sequence identity with any of SEQ ID Nos. 62 through 65.


SEQUENCE LISTING (66 sequences)

SEQ ID NO: 1 - protein, 657 aa, synthetic construct
MGHHHHHHSSGHIEGRHMASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDFEDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 2 - protein, 639 aa, synthetic construct
ASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 3 - protein, 680 aa, synthetic construct
MKHFLSLALCFSSINAVAVTVPHKSGGTGSPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQ

SEQ ID NO: 4 - protein, 680 aa, Fusarium graminearum; identical to SEQ ID NO: 3

SEQ ID NO: 5 - protein, 559 aa, Phanerodontia chrysosporium
MLSLLAVVSLAAATLAAPAASDAPGWRFDLKPNLSGIVALEAIVVNSSLVVIFDRATGDQPLKINGESTWGALWDLDTSTVRPLSVLTDSFCASGALLSNGTMVSMGGTPGGTGGDVAAPPGNQAIRIFEPCASPSGDGCTLFEDPATVHLLEERWYPSSVRIFDGSLMIIGGSHVLTPFYNVDPANSFEFFPSKEQTPRPSAFLERSLPANLFPRAFALPDGTVFIVANNQSIIYDIEKNTETILPDIPNGVRVTNPIDGSAILLPLSPPDFIPEVLVCGGSTADTSLPSTSLSSQHPATSQCSRIKLTPEGIKAGWQVEHMLEARMMPELVHVPNGQILITNGAGTGFAALSAVADPVGNSNADHPVLTPSLYTPDAPLGKRISNAGMPTTTIPRMYHSTVTLTQQGNFFIGGNNPNMNFTPPGTPGIKFPSELRIETLDPPFMFRSRPALLTMPEKLKFGQKVTVPITIPSDLKASKVQVALMDLGFSSHAFHSSARLVFMESSISADRKSLTFTAPPNGRVFPPGPAVVFLTIDDVTSPGERVMMGSGNPPPTLE

SEQ ID NO: 6 - protein, 558 aa, Pycnoporus cinnabarinus
MAPTAFSLVSALALASLSLAAPSAPGWSFDLKKETSGIVALEAIVVSPTLVVFFDRASDDPLQINNHSAWGALWNLESSTVRPLDVLTNSFCASGALLSNGTMASVGGDPDGFVGNPAIRPGNQAIRLFEPCDSPTGDGCTLFEDPATLHLLEKRWYPSSARIFDGSLIIVGGMHEATPFYNTDPALSFEFFPRKEDTPRPSEFLNRSLPANLFPRVFALPDGKVFMVANNQSIIYDIEAKTERILPDVPNNVRVTNPMDGSAILLPLSPPDFVPEVLVCGGSQTDTIDPSLLTSQTPASSQCSRIRLDEEGIAKGWEVEHMLEGRIMPELVHLPNGQVLIANGGRTGFAAIASVSEPVGNSNADHPVLVPSLYTPDAPLGRRISNVGLPSSGIPRLYHSSVTLTPQGNFLIAGSNPNNRTTVGPGIKFPSEFRVQTLDPPFMSVERPKILNMPKKLGFNKSFTVPISVPSSLARPGAKVQISLMDLGFSSHAFHSSARLVFMNGKISQDSKSLTFTTPPNGRVYPPGPATVFLTIDDVTSEGAWVMMGSGNSPPTLE

SEQ ID NO: 7 - protein, 506 aa, Colletotrichum graminicola
MPTLRSALRNLPAALLALAAACEAQNVGKWGPMVKFPVVPVAVALVPETGNLLVWSSGWPNRWTTAGNGKTYTSLYNVNTGNISDAIVQNTQHDMFCPGTSLDADGRIIVTGGSSAAKTSVLDFKKGESSPWTPLSNMQISRGYQSSCTTSEGKIFVIGGSFSGAGTRNGEVYDPKANTWTKLAGCPVKPLVMQRGMFPDSHAWLWSWKNGSVLQAGPSKKMNWYDTKGTGSNTPAGLRGTDEDSMCGVSVMYDAVAGKIFTYGGGKGYTGYDSTSNAHILTLGEPGQAVQVQKLANGKYNRGFANAVVMPDGKIWVVGGMQKMWLFSDTTPQLTPELFDPATGSFTPTTPHTVPRNYHSTALLMADATIWSGGGGLCGANCKENHFDGQFWSPPYLFEADGVTPAKRPVIQSLSDTAVRAGAPITITMQDAGAYTFSMIRVSATTHTVNTDQRRIPLDGQDGGDGKSFTVNVPNDYGVAIPGYYMLFAMNEAGVPCVAQFFKVTL

SEQ ID NO: 8 - protein, 497 aa, Colletotrichum gloeosporioides
EAEAQGLGQWSPLIKFPVVPVSVALLPESGNLLVWSSGWPNRWTTAGNGKTYTSLYNVQTGNVSDAVIQNTQHDMFCPGTSLDAEGRIIVTGGSSAAKTSVLDFKNGESSSWTALSNMQISRGYQSSCTTSEGKIFVIGGSFSGAGTRNGEIYDTATNKWTKLAGCPVKPLVMQLGMFPDSHAWLWSWKNGSVLQAGPAKQMNWYDTKGTGANTPAGLRGADQDSMCGVSVMYDAVAGKIFTYGGGKGYTGYQSTSNAHILTLGEPGQQVQVQKLQNGQYNRGFANAVVMPDGKIWVVGGMKQMALFSDATPQLTPELFDPATGKFTPTAAHTVPRNYHSTALLMADGTIWSGGGGLCGAGCAANKFDGQFWSPPYLFEADGKTPAKRPVIESLSDETVKAGAALTINMQDEGKYTFSMIRVSATTHTVNTDQRRIPLDGQDGGDGKSFSVNMPSDYGVVIPGYYMMFAMNEAGTPCVAKFFKVSLHHHHHHHHHHV

SEQ ID NO: 9 - protein, 711 aa, >Colletotrichum graminicola
MVRSCAYKAIAAASLLSQLASAAITSCPNNETVWETPIGVKYTLCPGSDYQNGGASLQTVRDIQSSLECAKICDSDARCNRAVYDNVNKACDVKNSTNPMQWAADDRFETIRLTNDLPEGAFISTCSFNETSYRVPETNAEYRICPDTDYTGVNAKVVEGVTTIQACAELCSNTQDCRKSVFDHINNACAIKAAEPATSIFWVQDKQFSTIRLPENIDPAVKGKWGDLIRLPVIPVAAYIVPSYPEPSRLLFFSSWSNDAFSGASGMTQFGDYDFATGAISQRTVTNTHHDMFCPGISQLEDGRILIQGGSDADTVSIYDPATNEFTRGPNMTLARGYQTSCTLSNGKVFTIGGAYSGERVGKNGEVYDPVANAWTYLPGADFRPMLTNDHEGIWREDNHAWLFGWKNGSIFQAGPSKDQHWYGIQGNGTVAKAATRDDDDAMCGVWVMYDAVAGKIFSAGGSPDYTDSPATQRAHITTIGEPNTPAEVERVADMGFPRGFANAVVLPDGQVLVTGGQRMSLVFTNTDGILVAELFNPETREWKQMAPMAVPRNYHSVSILLPDATVFSGGGGMCWVQNVGDSTAGCDKTVDHSDGEIFEPPYLFNEDGSRAARPVISAISADPIKAGATLTFTVEGVEGQGTAALIRLGSVTHSVNSDQRRVPLNVTVSGNEYSATLPDDYGILLPGYYYLFVSTPQGTPSIAKTVHVIL

SEQ ID NO: 10 - protein, 611 aa, Streptomyces lividans
STEKYHQYKINQPEYKAANGKWEIIEFPEKYRQNTIHAALLRTGKVLMVAGSGNNQDNSDDKQYDTRIWDPVKGTIKKVPTPSDLFCTGHTQLANGNLLIAGGTKRYEKLKGDVTKAGGLMVVHNENPDKPITLPAGTKFTGKENGKTFVSKDPVLVPRAEKVFDPATGAFVRNDPGLGRIYVEAQKSGSAYETGTEDNYRVQGLSGADARNTYGIAQKLALDKKDFQGIRDAFEFDPVAEKYIKVDPMHEARWYPTLTTLGDGKILSVSGLDDIGQLVPGKNEVYDPKTKAWTYTDKVRQFPTYPALFLMQNGKIFYSGANAGYGPDDVGRTPGVWDVETNKFTKVPGMSDANMLETANTVLLPPAQDEKYMVIGGGGVGESKLSSEKTRIADLKADDPKFVDGPSLEKGTRYPQASILPDDSVLVSGGSQDYRGRGDSNILQARLYHPDTNEFERVADPLVGRNYHSGSILLPDGRLMFFGSDSLYADKANTKPGKFEQRIEIYTPPYLYRDSRPDLSGGPQTIARGGSGTFTSRAASTVKKVRLIRPSASTHVTDVDQRSIALDFKADGDKLTVTVPSGKNLVQSGWYMMFVTDGEGTPSKAEWVRVP

SEQ ID NO: 11 - protein, 646 aa, Actinobacteria bacterium
MSELGGGRRRARRLAVGTVVVLALAGMNGPWVYRFSTEKYHEYAINRPEYKAENGKWDIIQFPKKYRQDTIHAVLLHTGKVLLIAGSGNDQDNFDAKKFDTRIWDPVKGTIKKVPTPADLFCTGHTQLANGNILIAGGTKKYEKLKGDVKKAGGVMILYNENPDKPITLPKGTVFTGKQNGKSFVSTDSSLIPRATKVFDKKTGKFLRNNPGYARVVVEAPKEGTTYETGTEDNYTIQGLTGADERNTYGIANKLGMDKRDFEGIKNAYEFDPVAEKYIKVDPMNEARWYPTLTTLSDGKVLSVSGLDDIGQLVPGKNEIFDPKTKTWTYTKTKRQFPTYPALFLMQNGKLFYSGSNAGYGPDDVGRDPGIWDVDTNAFTKLPGLSDPNMLETSATVLLAPAQEEKFMVIGGGGVGESKLSTNKTRLIDLKDANPKFVDGPTLEKGTRYAQASVLPDDSVLVSGGSQDYRGRSASDIKQARIYNPTTNTFKRVADPEVGRDYHSGSILLPDGRVMFFGSNPLYADKANTKPGVFEQRIEIYTPPYLYRDARPDLSGGPGTIARGASGTFTSKHASSIRKVRLIRPSSSTHVTDVDQRSIALDYTTSGDKITVTVPKNRNLVPSGWYMMFVDDDQGTPSKAVWVDVP

SEQ ID NO: 12 - protein, 666 aa, Streptomyces sp.
MTRQSGRGARRSAGPVSRRSGARARRSGLRTRRFAIGTVAVVALAGANGPWLYRFTTERYHHYTINTQSYKEANGHWDFLDIPAEFRVNAVHAALLRTGKVLLVAGSGNNEKNFDAKSFRSILWDPKTNAFKDIHTPKDLFCAGHTQLPGGNLLIAGGTKRYEKLGGDVTKAGGLMIVRNENPRKPITLPAGTRFTGKESGKTFISQDPLLVEKATKVFDKKTGEYQRNEAGVGRIYVEAEKSGRKYETGTEDNYRISGLSTEDTRNVYGIAQKLALDKKDFQGINDAYEFDPVAEKYIPVDPMDKARWYPTLTTLKDGKVIAVSGLDDIGQIDVGKAEIYDPKKKSWAPSGIVRKFATYPALFLLNDGKLFYSGSNSGYGLQSEGRTPGIWDLKTNDFQEVPGLVDADQMETSATVRLPPAQDERFMVIGGGGVGESDKSTPKSRLVDLQQKNPKFTEGASLDKGTRYPSASLLPDDSLLVAGGAGDYRGRGGSDVLEARLYDAKSDTYKRVADPAVGRNYHSGSVLLPDGRVMIFGSDPLYMDKINTRPGTFEQRIEIYTPPYLYRGSRPELTAGPKSIKRGGTGMFTTQHASKITSAKLMRPSAVTHVTDTDQRSIALELEKSADGITVTVPKNRALVPSGWYMLFVTDAKGTPSEGTWVEVP

SEQ ID NO: 13 - protein, 663 aa, Streptomyces abyssalis
MKYRPSRRTRRTAIGAAVVLVIAGFNGPALYGIASEKYHDYKINQPEYKAENGHWKTVQVPEKYRINTIHAALLHTGKVLLVAGSGNDAKQFNAKTFRTVLWDPVKNTFKNIPTPNDLFCAGHTQLPDGKLLVAGGTRRYEKLGGDVKKAGGLMIIHNENPDRAKTVKAGTRFTGRKNGKTFEAKDAVLVPRAKKKTNPATGKVEVTPSVARVYVEAIKRGKKYQTGTEDNYRLSGLRGAERRNIYGIAQKLSFDKKDFQGIKDTYEFDPVAERYLKTDFMDEARWYPTLTTLEDGKVLSVSGLDDIGQVVPGKNEIYDPQTKKWEFLPRKRFFPTYPALHLTDDNKIFYSGANAGYGPADKGRTPGLWNLETNGFRKIPGMSDPDKLETAMSVPLPPTQSRKFMVLGGGGVGESKKASEKTRIVDLKDPEPRFQDGPDLYAKARYPSSVILPNDKVLTTNGSGDYRGRSESNILKAEIYDPADNTKKPVADPIVGRNYHSGALLLPDGRVMTFGSDSLFSDKANTKPGKFEQQIDIYTPPYLHTKGERPELENTGRGEQTVRLGGTASFNSKDAGSIKKMRLVRPSSFTHVTNVEQSSVALKFKKSADGVTVKLPEDPSLVPPGWWMVTAVDDKGKPSKSVWLKVPRTASAQAPTTTPLSPP

SEQ ID NO: 14 - protein, 652 aa, Streptomyces indicus
MTVGSTPVKHTRRRARRIAIGAAVVLALAGMNGPALWRFASDQYHDYKINRPEYKADNGKWDIVELPDEYKLNTIHAALLHTGKVLLVAGSGNNQKNFDAKKFDTVLWDPVKNTYKKIPTPNDLFCTGHTQLPDGKLLVAGGTKSYEKLKGDVTKAGGVMNVYNENPDEPITLAKGTKLTGKENGKTFVITESIVVERAKKRFDKETGKFLGNTPGYARVYVEAEKRGKKYETGTQDNYKIVGLSGADAKNTYGIAQKLALDKKDFQGIDDAYEFDPVAEKYIKVDPMNEARWYPTLTTLEDGRVLALSGLDDIGQIVPGNRNEVYDPDTKKWTYVKQNRQFPTYPAIFLLPNGKLFYSGANAGYGPDDVGREPGIWDFDTNGFTKLKGLSDPKLMETAGTVWLPPAQDEKFMVVGGGGVGESEQSSDKTRIIDLSEDAPEFKDGPSFDKGTRYPQSSVLPDDSVLISGGSEDYRGRSGSNILEARMYDAKTGEMRRVADPLVGRNYHSGSLLLPDGRVVFFGSDSLFADEANTKPGEFEQRLEIYTPPYLYQGAQPTLTGGPDSIERGESATFKTQHASSIKNARLIRPSASTHVTDIDQRSIALDVKKGADSVTVTVPENKNLVQPGWYMLFVTDDQGVPSKAQWVEIPE

SEQ ID NO: 15 - protein, 645 aa, Streptomyces diastatochromogenes
MKDQAGRRRARRLAIGTAVVLALAGMNGPWLYRFGTEQYHQYKINKPEYKAANGHWDIVDFPEEYRQDTIHAALLHTGKVLLVAGSGNNQDNFDKKKFDTRIWDPVKGTIKKVPTPADLFCTGHTQLANGNLLIAGGTKRYEKLKGDVTKAGGLMVVHNENPDKPMTLPAGTKFTGKENGKTFVSKDPVLVPRAKKVFDPKTGAFLRNDPGLGRIYVEAQRSGTQYETGTQDNYRIQGLTGADARNTYGIAQKLALDKKDFQGIRDAYEFDPVAERYIKVDPMNEARWYPTLTTLSDGKILSVSGLDDIGQLVPGKNEIFDPKTKKWTYTQKIRQFPTYPALFLMQNGKIFYSGANAGYGPDNVGRVPGIWDVGTNKFTKLPGLSDPNMMETAGTVLLPPAQDEKFMVIGGGGVGESKLSSKKTRLIDLKAQNPHFVDGPQLEKGTRYPQSSILPDDTVLVSGGSEDYRGRGASNILQARMYHPDSNSFTQVADPLVGRNYHSGSILLPDGRVMFFGSDSLYADKANTKPGKFEQRIEIYTPPYLYRDSRPSLSGGPQTIARGASGTFTSKHASSIKKVRLIRPSASTHVTDVDQRSIALDFTTSGDKITVTVPKSRNLVQSGWYMLFVDDDQGTPSKAQWVKVP

SEQ ID NO: 16 - protein, 658 aa, Streptomyces carminius
MTYRPSRRFRRIAIASAVVAALAGANGPALYRFGGEAWHDHRINQPGYKAANGHWRTVDVPERYRVNTIHAALLHTGKVLLIAGSGNDAEQFRAKTFRTVLWDPEKNTFANIPTPKDLFCAGHTQLPDGRLLVAGGTQRYETLEGDVEKAGGLMTVHNEDPDKPVTLPAGTRFTGRANGKTFASQAPVVVPRAGKTTAPGTGKVTVTVSTARVYVEALKKGRRHQTGTQDQYRVHGLKGDDRRNVYGIAQKLSFDKKDFQGIRDSYEFDPVAERYVGVDPMNEARWYPTLTTLADGSVLSVSGLDEIGQVVPGRNEIYDPETQKWSYLPEERFFPTYPALFLAAGGRILYTGSNAGYGPADEGRTPGLWDLESNEFREVPGLGDPDVLETSMSVLLPPAQEQRYMVLGGGGVGESRRATARTGIVDLTEPEPRFTPGPDLYAPVRYPSSVILPDDTVLTTNGSGGYRGRGGSDVLRAGLYDARTDTARTVADPLVGRNYHSGALLLPDGRVMTFGSDSLFADKADTRPGEFQQQIDLYSPPYLFRGKRPELRGTGEPRTVELGGKVTFASEDAARVRTARLVRPGSFTHVTNVEQRSVALEFERTADGLTFTLPDDPSLVPPGYYMLNVLDGEGVPSESVWVKVPVSPDAEPVTVAAD

SEQ ID NO: 17 - protein, 647 aa, Streptomyces sp. Ru73
MARRRTRFKERKTWLGIGAVLVLAAFNAPAATGFAERVYHDYAINRDAYKAENGHWDVLDVPPEFRQNTIHAALLRTGKVLLVAGSGNNVKNFDAKRFTSVLWDPVRNSFKKIHTPNDLFCTGHIHLADGKLLFAGGTKRYEKLKGDVTKAGGLMIVHNENPNEPVRLPAGTVFTGRRNHRTFVAKDPIRIRPAKKVFDKETGRFLRTEASVDRIYVEARKKGRQYEAGAQDNYRVRGLTRARAHNVYGIATKLGLDKKDFQGIHDAYEFDPLAEKFTKVQSMHEARWYPTLVRLSDGRVLALSGLDDIGQVVPGKSEIYDPKSRTWAYSGKVRQFPTYPAVFPLSDGTLFYSGSNAGYGPADVGRTPGIWHFRTNTFTKLPGLDDAGLMETSGTVLLPPAQRQKFMVVGGGGIGESAKSSKKTRLIDLTQPHPSFHDGPSLDEGTRYPILSVLPDDTVLVTGGSKDYRGPVAENILQARLYDARSGTMRRVADPQVGRNYHSGAILLPDGRVLTFGGDSLFADKAKTQTGTFEQRLEVYTPPYLYRGARPALSHGPTTVGRGATAVFRTKDAAAVKTARLLHPSSTTHVTDVEQRSVALDFTRTKSGDGIEVTLPKNRNLVPSGWYMLFVTNRQGTPSTARWVKVP

SEQ ID NO: 18 - protein, 667 aa, Streptomyces sp.
MAHRPSRRPVRILLACGAVLALAAANAPAVYDFTSERYHAYEIDQADYKAEKGHWRTVDVPEKYRINTIHAALLNTGKVLLIAGSGNDAKQFDAKTFRTVLWDPKKNSFKEIDTPKDLFCAGHTQLPDGNLLVAGGTQRYETLKGDVQKAGGLMIVHNENPDKPKTLPEGTRFTGQENGKTFVSQASVLIPRAKKTVDKKTGKAKVTASSARVYVEAPEKGEKYQTGTQDQYRVEGLKGDEKQNIYGIAQKLSFDKKDFQGIKDSFEFDPVAERYITVDPMAEARWYPTLTTLEDGKVLSVSGLDEIGQVVPGKNEIYDPKSKKWKYLPKSRFFPTYPALFLTADSRIFFTGSNAGYGPDDQGRVPGLWDLDSNKFSEVPGLSDPDILETSMSVLLPPAQDQKFLVLGGGGVGESPKSTDRTGIADLKADKPRFTDGPDLYDKARYPSSVILPDDTVLTTNGSGDYRGKGASNVLKASLYDPGTNKFAEAAAPLVGRNYHSGGLLLPDGRVMTFGSDSLFSDKADSKPGKFEQTLEIYTPPYLHKGGERPELRDSDGARRTVKLGASADFRSKDAASIKKMRLIRPGAFTHVTNVEQRSIAMDFKRTADGVRVTVPHNPSLVPPGWYMLTAVDDRGVPSESVWLNVPVSKDAPTHKTTADAPAPAPN

SEQ ID NO: 19 - protein, 654 aa, Streptomyces sp.
MLPCCQASAALTARNPCLPANAHSHCSVSQAPELATLEYASPPVSTPYHDYTIIKPGYKAANGHWDLLDVPAGYRINTIHAALLHTGKVLLVAGSGNNRKNFDAKSFRSVLWDPRTGAFKNIPTPDDMFCSGHTQLPDGKLLIAGGTRRYEKLKGDVTKAGGLMIVHNENPDRPVTLPAGTRFTGRANGRTFVSKDPVLVEKATKVFDRRTGRFLHNDAGLGRIYVEAEKSGARYATGAEDNYRIAGLTGSDTRDVYGIAQKIALDKKDFQGIREAFEFDPVAEKYISVDPMNEARWYPTLTTLKDGRVLALSGLDEIGQIVPGKDEIYDPRTKKWRYTGIVRRFPTYPAVFLLGDGKLFYSGSNAGYGPADVGRDPGVWDLATNSFRKIPGLGDADETETSATVRLPPAQDERFMVIGGGGVGESDRSSAKSRLVGLKDPAPRFRDGAALAEGTRYPGVSLLPDDSVLVTGGSSDYRGRGGSNVLQARLYDPGTDRYRRVADPAVGRNYHSGSVLLPDGRVMIFGSDSLYADRANTRPGVFEQRIEIYTPPYLYRSSRPVLTGGPRSLRRGGTGGFTTAPGRAITSAKLIRPSAVTHVTDTDQRSVALGLRRTPGGITVTVPGNRALVPSGWYMLFVTDDRGTPSEGRWVEVP

SEQ ID NO: 20 - protein, 667 aa, Streptomyces mobaraensis
MRLRHLTDRRSGTVIGRRTRRTAVGAAVVLVLAGMNGPAIWGYASDQYHEYKINKPEYKAKNGHWDVVDVPDEYRINTIHAALLQTGKVLLVAGSGNNAANFAAKTFRTVLWDPERNTFKNVPTPKDLFCAGHTQLPDGKLLVAGGTQRYEKLEGDVTRAGGLMIVHNENPDKPMTFPAGTRFTGKQSGKTFESKDAVLVPRAKKEQGKDGKVKVTVSTARVYVEALEKGQEHATGLTDNYRIEGLTGDDARNFYGIANKLSFDKKDFQGIKDAFEFDPVAERYVTVDPMNEARWYPTLTTLQDGKVLSVSGLDEIGQVVPGKNEVYDPKTKKWTYLPQERFFPTYPALFLTDKGKIFYTGSNAGYGPADKGRDPGVWDLGNNSFIPVPGISDPDALETSMSVLLPPAQDQRYMVLGGGGVGEDKKSTARTRIVDLHTERPRFHDGPDLYAKARYPSSVILPDDTVLTTNGSGDYRGRSASNVLKAEIYDPKANASHRVADPLVGRNYHSGALLLPDGRVMTFGSDSLFRDKDNTQPGVFQQQIDLYTPPYLFHKGDRPEIRDTDRRIVKLGDKTTYRITSAHGVAKARLIRPGSFTHVTNIEQRSIALDLKKEGTDRFTVTLPKDPSLVPPGWYMVIVVDEEGTPSKAVWVKVPAAEKRDDEKKEE

SEQ ID NO: 21 - protein, 506 aa, Colletotrichum tofieldiae
MVTVCSVLRCLPAALLAFAAASEAQNVGQWGPMVKFPVVPVAVALLPETGNMLVWSSGWPNRWTTAGNGKTYTSMYDVKTGKVSDALIQNTQHDMFCPGTSMDADGRIIVTGGSSASKTSVLDFKKGESSSWAPLANMQISRGYQSSCTTSEGKIFLIGGSFSGAGRRDGEIYNPKANTWTKLPGCPVKPLVMQRGMFPDSHAWLWSWKNGYVLHAGPAKQMNWYDTKGTGANTPAGLRGADDDSMCGVSVMYDAVAGKIFTYGGGKAYTGVTSSSNAHILTLGEPGQAVQVQKLQNGKFNRGFANAVVMPDGKIWVVGGMRQMQLFSDNTPQLTPELFDPATGNFTPTTPHAVPRNYHSTALLMADATIWSGGGGLCGANCKENHFDGQFWSPPYLFEADGKTPAKRPVIQNLSETDVKAGAPITITMQDAGTYTFSMLRVSATTHTVNTDQRRIPLDGQDGGDGKDFTVNIPDDYGIAVPGYYMLFAMNEAGVPCVAKFFKVTL

SEQ ID NO: 22 - protein, 680 aa, Moelleriella libera RCEF 2490
MSRFLLLLALVAVASSAAVEQQQQQQQQPRAIKILEHYRESSTFTKLFAAAPIGNEIPKNAYTVTCDSYQPGNECALAIDGNNNTFWHTAFSGANLPHQIVVDLGATRNINGLSALPRQDGNNHGFIAQHEVAVSTDNRNWEVVASGTWYGGDSTTKFANFETRSIRYIRLKALSEAYGNQWTSVAEVKVYEAKTGPAAFAGTGKWGPTINFPTIPVAGAVDPLTGQVLIWSSYTYDNYLGSSQDRVFTSIWDPSTGVVTPKLVDNTNHDMFCPGISIDGAGRMVITGGNSAQKTTVYQFGSQTWIPGPDMNTQRGYQASATLSDGRVFTIGGCWSGGWFEKNGEVYDPKASTWTSLPGALVHPMLTNDAQGIYRADNHAWLFGWKNGSVFQAGPSTAMNWYTTSGNGSVAPAGNRTSSRGDAPDAMTGNAVMYDAVNGKILSFGGSPSYQDSSATTNAHIITIGSPGAPAQSRFASNGLWSPRAFHTSVILPDGKTFITGGQTYAVPFSDDNPDLTPEMYDPVADSFVQQQANTIIRVYHSISLLLPDGRVFNAGGGLCGDCTTNHFDGQIFTPQYLLTRTGQLAARPAINSVSLSGRRLTINTNSAITSAALMRYGSATHTVNTDQRRIPLKLTTTGTNRYTADAPSDPGILLPGYYMLFVLNSQGVPSVAKTVNFLV

SEQ ID NO: 23 - protein, 685 aa, Purpureocillium lilacinum
MKLLGTVAALALCDAASHTYAVAVKPRPHTAMPNLAARGTEMSLMAAKPIGNAINRAGWKVTCDGEEQGNECAKAIDGDNNTMWHTAWQNDNPPPPHTITVDMGSAQTINGISVLPRQDGSEHGWIARHDVLVSNDGQTWGDPVATGTWYTDATAKYANFEPRSARYVRLVARSEAQGRPWTSIAELNVYRADGPPVPKNGIGKWGLTLDFPVVPVAGVVDPLTGKVVVWSAYENDQYEGSPGGWTLTSTWDPATGEVTERNVTNIGHDMFCPGVSLDASGRVVVTGGSNAQKTSFYDAATEAWVPGPDMKTPRGYQASATCSDGRVFTIGGSWSGGQFEKNGEVWDPKTNSWRALPGAAVKPMLTKDRGGIYRADNHAWLFGWRNGSVFQAGPSTAMNWYYTAGDGRVRSAGQRRAPRGADPDAMCGNAVMFDATAGKILTVGGAPHYEDADATTNAHVLTLGDAGAAPKVVFAGNGMAHPRIFANAVVLPDGTVFVTGGQQHAELFKDTTPQLTPELYDPALGAFVEQAPNSVVRVYHSMALLLPDATVLSGGGGLCGGACDTNHFDAQVFSPRYLFDGEGQPAARPKIRAVASKEVHAGDAIKVTTDGPVKSAALVRYGSATHSVNTDQRRVPLTLRQDGGSYAYSADLPRDLGVLLPGYWMLFVMNDKGVPSVAATVKVLL

SEQ ID NO: 24 - protein, 671 aa, Penicillium antarcticum
MHAKWLSGLLLLGLSQAKDNSQALQTAATTNLQSAAEDPYATNQAPPIGEKLSRRRWTVTCDNSQTDECTKMLDDNFDTFWQSGNDGSIHEVVVDLSRAENIQALSIVSRRDTATTGQIFAHQIYVSNDKENWGNPVAFGTWYNTTDEKLAVFEPKEGQYLRLVSLDGNAASISELQIYDSDSPKQVSNGGVWGPTIDLPLVAVSGAVLPGTGQVLVWSSWAKDDYLHSTSQTLTALWDPKTGNVTQRTVQDTGHDMFCAGTSWDGNNELLVTGGNNDGQTSILDPATDTWYPGAAMKLGRAYHASATNSEGRVFIIGGSWNGGTNNNRDGEIYDPVAEEFTGLPGARVEPMWTNDANGGYRRDSHGWLFGWKNGTVFQAGPSKAMNWYYTSGQGDQKPAGNRGDADDAMSGNAVMFDAVNGKIISFGGSPSYENTYATKDAHLIEIGEPGTDPKVSTAKNPSGDGMAFARVFHTSVVLPNGNVFVAGGQTYATPFNDSGAQFTPEIYDPATNEFRQGQTNSVVRVYHSISLLLTDGRVFNGGSGLGVSAPSNHFDAQIYTPDYLLNNDGTLATRPTIDSVDKRTLHPGEKLIVKASIKLTKASLIRYGSTTHTMNTDQRRVVLDSWAANGETYETTLPSEAGALLPGPWMLFILNDDGVPSIAETIYIEV

SEQ ID NO: 25 - protein, 673 aa, Penicillium flavigenum
MRPRWLTGLLLLGLAHADNDNSVISDKQVANQDHSATTDPYATTQAPPRGEKLSRRRWSVTCDSSQSGNECEKLLDNDLQSFWLSGAEGNTHEVTIDLSRAENVHAISVVSRQDGKTDGQIAAHQVFVSGDKNDWGEPVAYGTWYEGTGEKLAIFEPKEGQYVRLVSLGSNVASISELNIYDGADPKTVPNGGVWGPTIDLPVVAVSGAVIPETNEVLVWSSWAKDDYLHSRGYTLTAVWNMNDNSVTQRKVQETHHDMFCSGMSYDGKGELLVTGGNNDKSTSIFDPASGNWTEGNTMIISRGYQASATLADGRVFIVGGSWNGGTNYDKDGEIYDPNTEEFRFLKNALVKPMWTDDYNSGYRRDSHGWLFGWKNDTVFQGGPSKNMNWYYTEGDGDQKPAGTRADASDSMSGNAVMFDAVNGKIITFGGSPSYENSYATTDAYLIEIDEPGSQPKVTAAKNPNGEGMAYARTFHTSVVLPDGGVFTAGGQSYGVPFNDSNAHLTPELYDPKTNQFNEQQPNSIVRVYHSISLLLPDGRVFNGGSGLGVSAPTNHFDAQIYSPHYLFNQDGSLATRPTIDGVVEKKLRAGDKLTISASIDVKNASLIRYGTTTHTVNTDQRRIALDSWTANGDSYETTLPGDNGILLPGPWMLFILNDDGVPSVSQTIHIQV

SEQ ID NO: 26 - protein, 684 aa, Fusarium sp. AF-8
MWSPILTLCLTGLFWDEAMAIVVPAQNSTKVKVQMEHLSMRATGPLGTAINRNNWKVTCDSDNPDSDNACQKAIDGDVNSFWHTAWFEDTSKDPGLPHTLTVDMKTVKNVNGISALPRQDGTTHGWIARHDIFLSTDGKTWGSPVATGTWYADGTEKFSNFETKRARYVRIVAITEAYDGPWTSIAEFNVYKAATYTAPKTGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRAFKPGEIFDPKSRKWTLLPNAKVEPMLTADAQGIFRSDNHAWLFGWKGETVFQAGPSSAMNWYYTQGKGTVKPAGKRQSSRGVDPDSMCGNAVMFDAAKGKIVTFGGTPNYQDSYATTNAHIITIGAPGTQASVAFVSDGMYYPRVFHTSVLLPDGTVFITGGQEYAIPFEDSTPQLTPELYIPDSDTFIKQQPNSIVRTYHSMSILLQDARVFNGGGGLCGDCSTNHFDAQIFTPSYLLTKDGKPAARPKIVSVSATTIKVGGSITVTTGGVVNTASLIRYGTATHTVNTDQRRIPLTLASAGKNKYTFKVPGDSGIALPGYWMLFVMNSAGVPSVATTIKVTT

SEQ ID NO: 27 - protein, 506 aa, Colletotrichum spinosum
MVTLCSTLRSLPAALLALAALSDAQGVGQWGPLVKFPIVPVAVALIPESGNMLVWSSGWPNRWTNAGNGKTFTSLYDVSTGKAGDAIVQNTQHDMFCPGTSLDVEGRIIVTGGSSAAKTSVLDFRKGESSSWAPLSNMQISRGYQSSCTTSEGKIFVIGGSFSGAGTRNGEIYDTASNKWTKLAGCPVKPLVMQRGLFQDSHTWLWSWKNGSVLQAGPAKQMNWYDTKGTGANTPAGLRGADDDAMCGVSVMFDAVAGKIFTYGGGKAYTGYAASSNAHILTLGEPGQQVQVQKLANGQYNRGFSNAVVLPDGRIWVVGGMRQMQLFSDNTPQLTPELFDPATGKFTPTAPHAIPRNYHSTALLMADATVWSGGGGLCGANCKENKFDGQFWSPPYLFEADGKTPAQRPVIDSLSDKTVRAGAPLVVTMKDEGKYTFSMLRVSATTHTVNTDQRRIPLDGQDGGDGKSFTVNMPGDYGVVIPGYYMMFAMNEAGVPCVAKFFKVAL

SEQ ID NO: 28 - protein, 684 aa, Pochonia chlamydosporia 123
MELFGAIAFVLCQLSCHVHAVTLKQTSTTALVHATEQAALHLLAAAPVGYRIDRANWKVTCDSQEIGYECGKAIDGDNSTVWHTAFRADNPKPPHNITVDMGSIQNVNGLSILPRQDSDSNGWVARHQVLVSADGQRWEHPVATGTWYADSTEKFSNFEPEQARFVRLVADSEVSGNPWTSVAELNVYKAGTDPAPAKSASSGKWGPTLDFPIVPVAAAVVPQSGKVLVWSAYENDKFEGSPGGYTLTSTWDPATGDVTQRNVTNIGHDMFCPGISMDSNGQIVVTGGNNAQKTSFYDAASDSWVPGPDMTVSRGYQSSATCSDGRIFTIGGSWSGGQFEKNGEIYDPSTKTWKMLSLAAVKPMLTADKKGRYRADNHGWLFGWRNGTVFQAGPSTAMNWYFTGGDGGVKSAGSRKSSRGPDPDSMCGNAVMFDATNGKILTVGGSPWYQDNDATANAHIITVRGPGSEPSVSFAGNGMHHARIFASSVVLPDGSVFITGGQQHSIPFADSTPQFTPELYDPEKDQFLEQAPNSIVRVYHSLALLLPDATVFNGGGGLCGGSCATNHFDAQIYSPRYLFDHDGSPAVRPVIKSVSRTSVKPGDSLSFTTDSAVQHASLVRYGTATHTVNTDQRRVPLTLDASGANTYAVQLPNDPGIMLPGYWMLFVMNERGTPSVAASIKAHL

SEQ ID NO: 29 - protein, 684 aa, Nectria haematococca
MKNLLTLSLCLGSLLDVTDAVVIPETPGPQPGKFKASPPLGIPIDRKGWTVTCSNQQSGNDCAKAIDGNTATFWQTNANTKAPVTITVDMKTARNVNGLSQLPLQDNSNNIGNWIERHSVFLSTDGKNWGSPVATGTWWTDKTEKFSNFEPQRARYVRLVAIPDETHDNPRISLAELNIYAASSNSNQEPGLGRWGPTLDFPVVPVASAVEPTSGQVVVWSARAYFEPAYRYDDFQHQNPRGGLTLSAVWDPATGVITQRNITLTHHDMFCPGISMDGDGQIVVTGGNDAKKTSLYDSPSDSWITGPEMNIARGYQSSATTSDGRVFTIGGSWNGPRGGKNGEIYNPDLKSWTLLPGALVKPMLTADKEGVYRSDNHGWLFGWKKGTVFQAGPSTAMNWYYTRGADGDVKPAGKRQVGDIVDPDSMCGNSVMYDAVKGKILTFGGSPNYRFSDSTANAHIITIGEPGSVAKTAFAGGGQGLHPRIFHTSVVLPDGTVFITGGQKHSEPFVDSTPQLEPEMYLPASDAFVKQQSNSIVRVYHSISLLLPDGRVFNGGGGLCGTCTTNHFDAQIFTPNYLFDKNGNLATRPRISSTSTKTAKVGSTITFTTNGPVKQGSLIRYGTATHTVNTDQRRIALTFTNTGTNRYSFKIPNDPGIALPGYWMLFVLNSAGVPSVATTIKVTN

SEQ ID NO: 30 - protein, 679 aa, Gibberella subglutinans
MKSFYSLALCLGAFFNAATAIDPEEQGQQPGKFAAAPPVGSNPIDRKGWTVKCSSQAPNFPCGRAIDGDKNTFWQTPYGTTNTPPPHTITIDMKQTQYVSGLQITPRQDGNTRNWIGRHEVYLSSDGTNWGKPVAFGTYWGDKYPWITNFETHPARYLRFVALSNVNSDYPWIAIADFQVYNALKYNPPAKGLGKWGPTIDFPVIPVAGAVEPVSGKVVIWSAYRYDAFQGTTPRGGFTLTSIWDPKTNVISNRNVSNNHHDMFCPGISMDGEGQIVVTGGNDAKKTTILMPDGNWVPGPDMQIARGYQSSATCSDGRVFTIGGSWSGARGGKNGEIYDPRAKTWTSLPKCLVGPMLTHDKEGVYKADNHAWLFGWKKGSVFQAGPSTAMNWYYTDRGTQGNTKAAGTRRKNGRVDPDSMNGNVAMFDAVKGKILTFGGATSYQQAPATANAHVLTIDQPGAIAQTALVGNNGAGIHARVFATSTILPDGNVFITGGQSYSNPFTDTNAQLEPEMFISSSNTFTKQQPNTIPRTYHSMSLLLPDGTVFNGGGGLCGSCKSNHFDAQIFTPQYLLDGNGNLATRPKITAVSATTAKVGSTITVTANSAIKSASLMRYGTATHVVNTDQRRIPLALTGAGTNKYSFKIPNDSGIALPGYWMLFVINNAGVPSVASTIKVTV

SEQ ID NO: 31 - protein, 681 aa, Gibberella subglutinans
MKSFWTLAFYLGNANAVAISQPASKAETPEGSLQFLSLRASAPIGTAINRDKWKVTCDSQHQGDECSKAIDGDRNTFWHTNWAAGATNDPKPPHTITIDMGSSQNVNGLSVLPRQDGSDHGWIGRHNVFLSTDGKNWGNAVATGTWFADITEKYSNFETRPARYVRLVAVTEANDQPWTSIAEINVFNAASYTSPQPGLGLWGPTLDFPIVPVAAAVEPTSGKVLVWSSYKNDAFGGSPGGVTLTSTWDPSTGVISQRTVTVTKHDMFCPGISMDGNGQVVVTGGNNAEKTSLYDSSSDSWIPGPDMKVARGYQFSATLSNGRVFTIGGSWSGGTFEKNGEVYDPSSKTWTSLPGALVKPMLTADQQGIYRSDNHGWLFGWKKGSVFQAGPSTAMNWYYTSGKGNTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCSTNHFDAQIYTPNNLYDSNGKLATRPKITNVSAKSAKVGGKITISTDTSIKQASLIRYGTSTHTVNTDQRRIPLSLRSTGSGNSYSFQVPSDSGIALPGYWMLFVMNSAGVPSIASTLLVTQ

SEQ ID NO: 32 - protein, 681 aa, Gibberella moniliformis
MKSFWTLAFYLGGASAVAISQPAAKSETPAGSLQFLSLRASAPLGTAINRDKWKVTCDSQHEGDECSKAIDGDRNTFWHTNWAAGGTNDPKPPHTITIDMGSSQNVNGLSVLPRQDGSNHGWIGRHNVFLSTNGKNWGDAVATGTWFADNTEKYSNFETRPARYVRLVAVTEANDQAWTSIAEINVFKAASYTSPQPGLGRWGPTLDFPIVPVAAAVEPTSGKVLVWSSYKNDEFGGSPGGVTLTSTWDPSTGVISQRTVTNTKHDMFCPGISMDGNGQVVVTGGNNAEKTSLYDSSSDSWIPGPDMKVARGYQSSATLSNGRVFTIGGSWSGGISEKNGEVYDPSSKTWTSLSGALVKPMLTADQQGLYRSDNHGWLFGWKKGSVFQAGPSTAMNWYYTSGNGNTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSVSLLLPDGRVFNGGGGLCGGCTTNHFDAQIYTPNNLYDSNGKLATRPKITKVSAKSVKVGGKITISTDSSIKQASLIRYGTSTHTVNTDQRRIPLSLRSTGSGNSYSFQVPSDSGIALPGYWMLFVMNSAGVPSVASTLLVTQ

SEQ ID NO: 33 - protein, 680 aa, Fusarium coffeatum
MRHLLTLALCFTSINAVAITKSHKAAGTGEPEGSFQFLSLRASAPIGTTISRDKWSVTCDSFHDGEGCDKAIDGDLNTFWHSQWGNGNDPKPPHTYTIDMGSTQNVNGLSVLPRQDGSSNGWIGRHEVFLSSDGKNWGSAVATGTWYADSTTKYSNFETRPARYVRLVALTEAKGQPWTSIAEINVFKADSYSAPRAGLGRWGPTLDFPIVPAAAFVEPTSGKVVVFSSYRNDAFGGSPGGITLTSSWDPANNVIAERTVTVTKHDMFCPGISMDGAGQIVVTGGNDAKKTSLYDSSSDSWIPGPDMQNARGYQSSATTSDGRVFTIGGSWSGGIFEKNGEIYNPSSKTWTSLSGAKVNPMLTADRQGLYRSDNHAWLFGWKKGIVFQAGPSTAMNWYYTSGNGDVKSAGKRQSNRGVATDAMCGNAVMYDAVQGKILTFGGAKDYQDTDATTDAHIITLGEPGTTPKTVYASNGLWYPRTFHTSVVLPDGSTFITGGQVRGIPFEDSTPQLTPELYVPGDDTFYKQQPNSIVRVYHSVSLLLPDGRVFNGGGGLCGDCTTNHFDAQVFTPNYLYDNNGNLVTRPKITNTSTKSVKIGGRVTITTDGSIQKASLVRYGTATHTVNTDQRRIPLTLSNSGRNSYSFTVPNDSGVALPGYWMLFVMNSAGVPSVSTTIRITS

SEQ ID NO: 34 - protein, 680 aa, Fusarium sporotrichioides
MKHLLTLALCFSSINAVAITNPHKTAGHDHPEGSLQFLSLRASAPIGSAISRNNWAVTCDSAQSGNECNKAIDGNQDTFWHTFYGANGDPKPPHTYTIDMKSTQNVNGLSMLPRQDGSRNGWIGRHEVYLSTDGTNWGSPVAAGSWFADSTTKYSNFETRPARYVRLVAVTEASGQPWTSIAEINVFQASSYTAPQPGLGRWGPTIDLPIVPAAAAVEPTSGRVLVWSSYRNDAFGGSPGGVTLTSSWDPSSGIVSDRTVTVTKHDMFCPGISMDGNGQIVVTGGNDAKKTSLYDSSSDSWIPGPDMQVARGYQSSATMSDGRVFTIGGSWSGGIFEKNGEVYSPSSKTWTSLPNAKVNPMLTADKQGVYRSDNHAWLFGWKKGSVFQAGPSTAMNWYYTSGSGDVKSAGKRQSNRGVAPDAMCGNAVMYDAVRGKILTFGGSPDYQDSDATANAHIITLGEPGSTPNTVFASNGLYFARTFHTSVVLPDGSTFITGGQRRGIPFEDSTPVFTPEVYVPEQDTFYKQNPNSIVRVYHSISLLLPDGRVFNGGGGLCGDCTTNHFDAQIFTPNYLYDGNGNLATRPKITRTSTQSVKVGGRVTISTDSSIVKASLIRYGTATHTVNTDQRRIPLTLTNNGGNSYSFQVPSDSGIALPGYWMLFVMNSAGVPSVAATIRVTQ

SEQ ID NO: 35 - protein, 682 aa, Fusarium longipes
MKQLLTLALCFSSINAVAINPHGHHNNKGTGDHEGSLQFLSLRASAPLGSAIARENWVVTCDSAQPGNECNKAIDGDTNTFWHTFYGNNGDPKPPHTYTIDMGSNRNVNGLSVLPRQDGNRNGWIGRHEVYLSTDGSNWGSPVAYGSWFADSTTKYSNFETRPARYVRLVALTEASGQPWTSIAEINVYQAGSYTAPQAGLGRWGPTIDLPIVPAAAAIEPTSGRVLMWSSYRNDAFGGSPGGITLTSSWDPSSGIVSDRTVTVTNHDMFCPGISMDGNGQIVVTGGNDAKKTSLYDSSSDSWIPGPDMQVARGYQSSATMSDGRVFTIGGSWSGGIFEKNGEVYSPSSKTWTSLPGAKVNPMLTADKQGLYRSDNHAWLFGWKKGSVFQAGPSTAMNWYYTSGSGDVKSAGRRQSNRGLDPDAMCGNAVMYDAVAGKILTFGGSPDYQDSDATTNAHIITLGEPGSTPNTVFASNGLYFPRTFHTSVVLPDGSTFITGGQRRGIPFEDSTPQLTPEIYVPEQDTFYKQNPNSIVRVYHSISLLLPDGRVFNGGGGLCGDCTTNHFDAQIFTPNYLYNSNGDLATRPKITRTSAQSVRVGGRITISTDSSIRRASLIRYGTATHTVNTDQRRIPLTLTNNGGNSYSFQVPSDSGIALPGYWMLFVMNSAGVPSVATTLRVTR

SEQ ID NO: 36 - protein, 645 aa, Streptomyces lividans
MKDRAGRRRARRFAIGTAVVVALAGMNGPWLYRFSTEKYHQYKINQPEYKAANGKWEIIEFPEKYRQNTIHAALLRTGKVLMVAGSGNNQDNSDDKQYDTRIWDPVKGTIKKVPTPSDLFCTGHTQLANGNLLIAGGTKRYEKLKGDVTKAGGLMVVHNENPDKPITLPAGTKFTGKENGKTFVSKDPVLVPRAEKVFDPATGAFVRNDPGLGRIYVEAQKSGSAYETGTEDNYRVQGLSGADARNTYGIAQKLALDKKDFQGIRDAFEFDPVAEKYIKVDPMHEARWYPTLTTLGDGKILSVSGLDDIGQLVPGKNEVYDPKTKAWTYTDKVRQFPTYPALFLMQNGKIFYSGANAGYGPDDVGRTPGVWDVETNKFTKVPGMSDANMLETANTVLLPPAQDEKYMVIGGGGVGESKLSSEKTRIADLKADDPKFVDGPSLEKGTRYPQASILPDDSVLVSGGSQDYRGRGDSNILQARLYHPDTNEFERVADPLVGRNYHSGSILLPDGRLMFFGSDSLYADKANTKPGKFEQRIEIYTPPYLYRDSRPDLSGGPQTIARGGSGTFTSRAASTVKKVRLIRPSASTHVTDVDQRSIALDFKADGDKLTVTVPSGKNLVQSGWYMMFVTDGEGTPSKAEWVRVP

SEQ ID NO: 37 - protein, 730 aa, unidentified
MRHFWLLPAVAGIAGAQCPYLSGEMSFTQEQDNAGDTIEVTEQPIDNTLYVNDTGSYMTTDFGTPISDQTSLKAGPRGPTLLEDFIFRQKLQRFDHERVPERVVHARGAGAYGTFKSYADWSNVTAADFLSANDKETPMFCRFSTVVGFRGSVDTARDVHGHACRFYTDEGNYDIVGINFAPFFIQDAIQFPDLVHAIKPMPNNEIPQAATAHTSAWDFFSQQSTALHSALWLMSGNGIPRSFRHMNGYGVHSFRFVAANGTSKVVRTPWKSQQGVASLVWDEAQAAAGKNSDYHRQDLYNAMPNGHYPKYELQAQIMDEADMLRFGFDLLDPTKLVPEEVVPYTPLGMMELNANPTNYFAEVEQAGFQPGHVVPGIDFTDDPLLQGRLFSYLDTQLTRHGGPNFEQIPVNRPRKPVHNNNRDGFGQQQIPTNNWAYTPNSMSNGYPMQANQTQGHGFFTAPYRYASGHLVRQTSPTFNDHWSQPAMFWNSLIPAEQQMVVNAIVFENSKVNSPHVRKNVVNQLNMVNNNLAVRVARGLGLDEPSPNPTYYTSNKTSNVGTFGKPLLSIEGLQVGFLASNSHPESIKQGQAMAAQFSAAGVDLNIVTEAYADGVNTTYALSDAIDFDALIIADGVQSLFASPALANQMNSTATSTLYPPARPFQILVDSFRYGKPVAAVGSGSVALKNAGIDSSRSGVYTGSSETTEKIAKEVLEGLYTFRFVDRFALDE

SEQ ID NO: 38 - protein, 730 aa, unidentified; identical to SEQ ID NO: 37

SEQ ID NO: 39 - protein, 503 aa, Micrococcus luteus
MEHQKTTPHATGSTRQNGAPAVSDRQSLTVGSEGPIVLHDTHLLETHQHFNRMNIPERRPHAKGSGAFGEFEVTEDVSKYTKALVFQPGTKTETLLRFSTVAGELGSPDTWRDVRGFALRFYTEEGNYDLVGNNTPIFFLRDPMKFTHFIRSQKRLPDSGLRDATMQWDFWTNNPESAHQVTYLMGPRGLPRTWREMNGYGSHTYLWVNAQGEKHWVKYHFISQQGVHNLSNDEATKIAGENADFHRQDLFESIAKGDHPKWDLYIQAIPYEEGKTYRFNPFDLTKTISQKDYPRIKVGTLTLNRNPENHFAQIESAAFSPSNTVPGIGLSPDRMLLGRAFAYHDAQLYRVGAHVNQLPVNRPKNAVHNYAFEGQMWYDHTGDRSTYVPNSNGDSWSDETGPVDDGWEADGTLTREAQALRADDDDFGQAGTLVREVFSDQERDDFVETVAGALKGVRQDVQARAFEYWKNVDATIGQRIEDEVKRHEGDGIPGVEAGGEARM

SEQ ID NO: 40 - protein, 527 aa, Bos taurus
MADNRDPASDQMKHWKEQRAAQKPDVLTTGGGNPVGDKLNSLTVGPRGPLLVQDVVFTDEMAHFDRERIPERVVHAKGAGAFGYFEVTHDITRYSKAKVFEHIGKRTPIAVRFSTVAGESGSADTVRDPRGFAVKFYTEDGNWDLVGNNTPIFFIRDALLFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLAHEDPDYGLRDLFNAIATGNYPSWTLYIQVMTFSEAEIFPFNPFDLTKVWPHGDYPLIPVGKLVLNRNPVNYFAEVEQLAFDPSNMPPGIEPSPDKMLQGRLFAYPDTHRHRLGPNYLQIPVNCPYRARVANYQRDGPMCMMDNQGGAPNYYPNSFSAPEHQPSALEHRTHFSGDVQRFNSANDDNVTQVRTFYLKVLNEEQRKRLCENIAGHLKDAQLFIQKKAVKNFSDVHPEYGSRIQALLDKYNEEKPKNAVHTYVQHGSHLSAREKANL

SEQ ID NO: 41 - protein, 502 aa, Corynebacterium glutamicum
MRPKLSGNTTRHNGAPVPSENISATAGPQGPNVLNDIHLIEKLAHFNRENVPERIPHAKGHGAFGELHITEDVSEYTKADLFQPGKVTPLAVRFSTVAGEQGSPDTWRDVHGFALRFYTEEGNYDIVGNNTPTFFLRDGMKFPDFIHSQKRLNKNGLRDADMQWDFWTRAPESAHQVTYLMGDRGTPKTSRHQDGFGSHTFQWINAEGKPVWVKYHFKTRQGWDCFTDAEAAKVAGENADYQREDLYNAIENGDFPIWDVKVQIMPFEDAENYRWNPFDLTKTWSQKDYPLIPVGYFILNRNPRNFFAQIEQLALDPGNIVPGVGLSPDRMLQARIFAYADQQRYRIGANYRDLPVNRPINEVNTYSREGSMQYIFDAEGEPSYSPNRYDKGAGYLDNGTDSSSNHTSYGQADDIYVNPDPHGTDLVRAAYVKHQDDDDFIQPGILYREVLDEGEKERLADNISNAMQGISEATEPRVYDYWNNVDENLGARVKELYLQKKA

SEQ ID NO: 42 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 43 - protein, 353 aa, Armoracia rusticana
MHFSSSSTLFTCITLIPLVCLILHASLSDAQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSLLHDMVEVVDFVSSM

SEQ ID NO: 44 - protein, 308 aa, Armoracia rusticana
QLSPSFYDKTCPQVFDIATNTIKTALRSDPRIAASILRLHFHDCFVNGCDASILLDNTTSFRTEKDAFGNARSARGFDVIDTMKAAVEKACPKTVSCADLLAIAAQKSVVLAGGPSWKVPSGRRDSLRGFMDLANDNLPGPSSTLQVLKDKFRNVGLDRPSDLVALSGGHTFGKNQCQFIMDRLYNFSNSGKPDPTLDKSYLSTLRKQCPRNGNLSVLVDFDLRTPTIFDNKYYVNLKENKGLIQSDQELFSSPDASDTIPLVRAYADGQGKFFDAFVEAMIRMGNLSPSTGKQGEIRLNCRVVNSKP

SEQ ID NO: 45 - protein, 308 aa, Armoracia rusticana; identical to SEQ ID NO: 44

SEQ ID NO: 46 - protein, 376 aa, Streptomyces lividans
ATPLTSLGSEQAMFHGKHQPGITTPMQARGHLVAFDLAAGAGRKEAAALLRRWSDTARRLMAGEPAGSRDTDVARDAGPSSLTVTFGFGHSFFGRTGLEKQRPVALDPLPDFSSDHLDKNRSNGDLWVQIGADDALVAFHALRAIQRDAGAAARVRWQMNGFNRSPGATAHPMTARNLMGQVDGTRNPKPGEADFDRRIFVPEEPEAGKGGPAWMANGSYVVVRRIRMLLDDWEELSLKAQEDVIGRRKSDGAPLSGGSGATESTEMDLEKTDGSGELVVPINAHARITRPDQNGGAAMVRRPFSYHDGFDADGVPDAGLLFVCWQADPLRGFVPVQRKLDRGDALSQFIRHEASGLFAVPGGAAEGEYVGQRLLE

SEQ ID NO: 47 - protein, 712 aa, Bos taurus
MWVCLQLPVFLASVTLFEVAASDTIAQAASTTTISDAVSKVKIQVNKAFLDSRTRLKTTLSSEAPTTQQLSEYFKHAKGRTRTAIRNGQVWEESLKRLRRDTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 48 - protein, 372 aa, Leptoxyphium fumago
MFSKVLPFVGAVAALPHSVRQEPGSGIGYPYDNNTLPYVAPGPTDSRAPCPALNALANHGYIPHDGRAISRETLQNAFLNHMGIANSVIELALTNAFVVCEYVTGSDCGDSLVNLTLLAEPHAFEHDHSFSRKDYKQGVANSNDFIDNRNFDAETFQTSLDVVAGKTHFDYADMNEIRLQRESLSNELDFPGWFTESKPIQNVESGFIFALVSDFNLPDNDENPLVRIDWWKYWFTNESFPYHLGWHPPSPAREIEFVTSASSAVLAASVTSTPSSLPSGAIGPGAEAVPLSFASTMTPFLLATNAPYYAQDPTLGPNDKREAAPAATTSMAVFKNPYLEAIGTQDIKNQQAYVSSKAAAMASAMAANKARN

SEQ ID NO: 49 - protein, 378 aa, Phanerodontia chrysosporium
MAFKSLIAFVALAAAVRAAPTAVCPDGTRVSHAACCAFIPLAQDLQETIFQNECGEDAHEVIRLTFHDAIAISRSQGPKAGGGADGSMLLFPTVEPNFSANNGIDDSVNNLIPFMQKHNTISAADLVQFAGAVALSNCPGAPRLEFLAGRPNKTIAAVDGLIPEPQDSVTKILQRFEDAGGFTPFEVVSLLASHSVARADKVDQTIDAAPFDSTPFTFDTQVFLEVLLKGVGFPGSANNTGEVASPLPLGSGSDTGEMRLQSDFALAHDPRTACIWQGFVNEQAFMAASFRAAMSKLAVLGHNRNSLIDCSDVVPVPKPATGQPAMFPASTGPQDLELSCPSERFPTLTTQPGASQSLIAHCPDGSMSCPGVQFNGPA

SEQ ID NO: 50 - protein, 430 aa, Thermobifida fusca DyP
MTEPDTERKGSSRRGFLAGLGAAALTGAGIGMAAGEVLRPLLPDSDPAASPKAEQRLRMAAQRADATAAPQPGISGPAPAFVHVIALDLAEEARKNPDTARDSAAAALRSWTELAARLHEESPHDIAEGAASAGLLPASLMVTVGIGGSLLSAIDAEDRRPDALADLPEFSTDDLHPRWCGGDFMLQVGAEDPMVLTAAVEELVAAAADATAVRWSLRGFRRTAAAARDPDATPRNLMGQIDGTANPAQDHPLFDRTITARPADNPAHAWMDGGSYLVVRRIRMLLTEWRKLDVAARERVIGRRLDTGAPLGSRNETDPVVLSARDEEGEPLIPENAHVRLASPENNLGARMFRRGYSYDQGWRDDGVRDAGLLFMAWQGDPATGFIPVQRSLADQGDALNRYIRHEGSALFAVPAAREGRYLGQDLIEG

SEQ ID NO: 51 - protein, 403 aa, Saccharomonospora viridis
MKGRRFGRRAFLATGAAALGGAAALGGRELFTGDGGADGRDRPDRGDIGRATVDFHGERQAGVATPAQAFATFVAFDLLDGVDREALIRWMRVWTDDIERLTRGAPALTDTEPELALLPARLTVTVGFGPGFLAAAGREELRPSWLAPLPEFPIDRLREEFSGGDLVAQVCADDEVTVAHAVRVLTKQARSFARPRWVQRGFRNTPGAVPEGATMRNLMGQLDGTRNLRPGPDDRLIWISDGPEWLRGGTGMVVRRIAMNLDTWDELDRPARELVIGRRLDNGAPLTGRHEHDEPDLEAVDERGLSVIPMFAHIRRARSDNPDERFLRRSYNYDDPPEPGELSNSGLVFVTFQADIEAQFTPIQRRLAELDSLNDWTTPIGSAVFAVPRGCRPGEYLGQPLLE

SEQ ID NO: 52 - protein, 299 aa, Escherichia coli
MSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 53 - protein, 343 aa, Cellulomonas bogoriensis
MTPPPSFIALVALDLASTSDRASVERLLRVWTVDIERLTTGRPGLADSEPELALVPAALTVTVGFGPGLLTAAGLRHRAPAWLHPLPPFGIDRLDPAWCDGDVVLQVCADDRTTLAHAVRVLTKEAQGLASVRWVQRGFRRSPGISEPDGTSMRNLMGQVEGTANLDPRTDPDLLWHRDGEPGWLTGGTSMVVRRIAMNLDTWDELSRGAREATIGRTLRTGAPLTGRAEHDEPDLEALDDHGRPVIDLEAHIRRARPTQREETFLRRAYNYDEAPPPGRASDSGLLFVTYQRDVDAQFTPVQRRLDAADLLNEWTFPVGSAVFAVPGGWSAGEYVGQRLLEG

SEQ ID NO: 54 - protein, 427 aa, Thermobifida cellulosilytica
MTGPEPTPAGSSRRGFLAGLGAAALTGAGLGMAAGEAVRPLLPDSDSEQPDPVDAQRLDMARRRADANAAPQPGISGRAPAFVHVIAFDLAEPARAEPAAAREGAATALRTWAEHAARLHADGPEGAASAGLLPASLMVTIGIGGSLLEAMDAADRRPDALADLPEFATDDLRPRWCGGDLMLQVGAEDPMVLAAAVDELVAATAPTTTVRWSLRGFRRTAAAAQDPDATPRNLMGQIDGTANPAQDHPLFTRTVTAPPADDPAHAWMDGGSYLVVRRIRMLLDEWRRLDVPDRERVIGRHLDTGAPLGGEKETDPVVLTARDADGRLVIPEDAHVRLANPENNLGARMVRRGYNYDEGWRDDGVRDAGLLFMAWQGNPATGFVPVQRSLVEQGDALNRYTRHEGSALFAVPAATADRYPGQDLVEG

SEQ ID NO: 55 - protein, 316 aa, Streptomyces coelicolor
MGGEVEEPEPQMVLSPLTSAAIFLVVTIDSGGEDTVRDLLSDVASLERAVGFRAQPDGRLSCVTGIGSEAWDRLFSGARPAGLHPFRELDGPVHRAVATPGDLLFHIRASRLDLCFALATEIMGRLRGAVTPQDEVHGFKYFDERDMLGFVDGTENPTGAAARRAVLVGAEDPAFAGGSYAVVQKYLHDIDAWEGLSVEAQERVIGRRKMTDVELSDDVKPADSHVALTSVTGPDGSDLEILRDNMPFGSVGREEFGTYFIGYARTPEVTETMLERMFLGTASAPHDRILDFSTAVTGSLFFTPAADFLEDLPARP

SEQ ID NO: 56 - protein, 138 aa, Amphitrite ornata
MGFKQDIATIRGDLRTYAQDIFLAFLNKYPDERRYFKNYVGKSDQELKSMAKFGDHTEKVFNLMMEVADRATDCVPLASDANTLVQMKQHSSLTTGNFEKLFVALVEYMRASGQSFDSQSWDRFGKNLVSALSSAGMK

SEQ ID NO: 57 - protein, 261 aa, Thermothelomyces thermophilus
MRASVLPVLIAISPALAGFDTWSPPGPYDVRAPCPMLNTLANHGFLPHDGKDITREQTENALFEALHINKTLASFLFDFALTTNPKNTSTFSLNDLGNHNILEHDASLSRADAYFGNVLQFNQTVFDETKTYWEGDTIDLRMAAKARLGRIKTSQATNPTYSMSELGDAFTYGESAAYVVVLGDKESRTVKRSWVEWFFEHEQLPQHLGWKRPAASFEEEDLNSSMEEIEKYTKELEGSNSTSGSQKHRRRLPRRRAHFGF

SEQ ID NO: 58 - protein, 504 aa, PLEUROTUS SAPIDUS
MTTPAPPLDLNNIQGDILGGLPKKTETYFFFDVTNVDRFKANMTQFIPHVKTSAGIVKDREAIKEHKRQKRPGLVPMAAVNVSFSHLGLQKLGITDDLSDSSFTTGQRKDAEVLGDPGTKNGDTFTPAWEAPFLKDIHGVIFVAGDCHASVHKKLDEIKHIFGVGTSHASISEVTHVRGDVRPGQVSAHEHFGFLDGISNPAVDQFDQNPFPGQDSIRPGFILAKENGDSRAAARPDWAKDGSFLTFRYLFQMVPEFDDFLESNPIVLPGLSRKEGSELLGARIVGRWKSGAPIEITPLKDDPKLGADAQRNNNFDFGDSLVRGDQTKCPFAAHIRKTYPRNDLEGPPLNADIDNRRIIRRGIQFGPEVTSQEHHDKKTHHGRGLLFVCYSSSIDDGFHFIQQSWANAPNFPVNAVTSAGPIPPLDGVIPGFDAIIGQKVGGGIRQISGTNPNDPTTNITLPDQDFVIPRGGEYFFSPSISALKTKFAAGVASSAPQAQAPIST

SEQ ID NO: 59 - protein, 480 aa, Irpex lacteus
MHVKRARSTPLIGSFPGQPPLPTIAQVQSTSAGNDSLPFENIQGDILVGMKKDKEKFVFFHINNATAFKSVLKTYAPANITSVATIIGPVANQPLAFVNLAFSHAGFGALNVTDDLQDTAFSDGQFKDSPNLGDDTSTWEEAFKGTNVDGVFLIGSNDESITAQYRDDLNAKFGDAWTIVYDLDSAARPGNEKGHEHFGYLDGISNPTIPGFGTPHPGQAVVDPGIIFTGRSKDPVMNRPSWALDGSFLVFRKLKQLVPEFNKYVLDNALQNQAGNLTVEEGAELLGSRMFGRWKSGAPIDLSPDFDDPALGNDIERNNNFNYSHPGSDLATDQTRCPFTAHIRKTNPRDLEGQGLFGDTFHAIRAGTPYGPEVTDYEASSNTTTIDRGLAFVEYQSVIGNGFRFQQQAWANNPRFPFSKGPSIQLGLDPVIGQGSPRETFGLDPRNASESFTVPQVIISNGGEYFFSPSITAIVEKFAA

SEQ ID NO: 60 - protein, 250 aa, Nicotiana tabacum
MGKCYPTVSEEYLKAVDKCKRKLRGLIAEKNCAPLMLRLAWHSAGTYDVCSKTGGPFGTMRLKAEQGHGANNGIDIAIRLLEPIKEQFPILSYGDFYQLAGVVAVEVTGGPDVPFHPGREDKTEPPVEGRLPDATKGSDHLRDVFVKQMGLSDKDIVALSGGHTLGRCHKERSGFEGPWTTNPLIFDNSYFTELLSGEKEGLLQLPSDKALLSDPAFRPLVEKYAADEDAFFADYAEAHLKLSELGFAEA

SEQ ID NO: 61 - protein, 519 aa, Trametes versicolor
MGLQRFSFFVTLALVARSLAAIGPVASLVVANAPVSPDGFLRDAIVVNGVVPSPLITGKKGDRFQLNVDDTLTNHSMLKSTSIHWHGFFQAGTNWADGPAFVNQCPIASGHSFLYDFHVPDQAGTFWYHSHLSTQYCDGLRGPFVVYDPKDPHASRYDVDNESTVITLTDWYHTAARLGPRFPLGADATLINGLGRSASTPTAALAVINVQHGKRYRFRLVSISCDPNYTFSIDGHNLTVIEVDGINSQPLLVDSIQIFAAQRYSFVLNANQTVGNYWVRANPNFGTVGFAGGINSAILRYQGAPVAEPTTTQTTSVIPLIETNLHPLARMPVPGSPTPGGVDKALNLAFNFNGTNFFINNATFTPPTVPVLLQILSGAQTAQDLLPAGSVYPLPAHSTIEITLPATALAPGAPHPFHLHGHAFAVVRSAGSTTYNYNDPIFRDVVSTGTPAAGDNVTIRFQTDNPGPWFLHCHIDFHLDAGFAIVFAEDVADVKAANPVPKAWSDLCPIYDGLSEANQ

SEQ ID NO: 62 - protein, 559 aa, Phanerodontia chrysosporium; identical to SEQ ID NO: 5

SEQ ID NO: 63 - protein, 928 aa, Myceliophthora thermophila
MTNAACLSYCASKGFPYAGTEYSVECFCGTTLASSSAKVADSECNMPCSGAPSEPCGAGSRLSLFHSSAVTGPAANPGVNDFTHLGCYAEGKTGRALTYNPGLPGADMTVAKCTAACRAANYILAGVEYGGECYCGNTIANGGAPADSGCSMVCNGNSTEFCGGPDRLNVYSYKNQYEPTATSTTGAGSTSSSSVPSATGLPEGWSYQGCWIDGKQGRILPYQLPDSQTNSRAACANACAEAGYTVSGTEYAVQCFCGDAIHNGGVETDEADCSTPCPGAPGEKCGAGDRLSIVSRGPPKIYAPPAPIEKIGDWEYQGCAEDNINDKRTFFWQIFFNDIMTPEMCLDRCAEFGYHAAGLEYGQECYCGDPANMATHGATFRPESECNVVCAGNSTAICGGLARLTTYFWIGTPFYSWDFPQDWRAGKYEFLVDGVNIPLITHETITGKVSFISKGATGPGNETGAYEFDPATLEFRELHIKTDVFCAASVTLPDKAGRQLNVGGWAGEATYGTRLYWPDGAPGVPGTHDWQENVNVLHLQAGRWYPSVLVLTNGSVMVVGGLIGSNDAATPSIEILPYTGTPPLYMDWLDRTHPNNLYPFLCILPGGGIFVQYWNEARILDPVTFDTVKTLPDAPGAPNDPKGGRTYPLEGTAVLLPQKYPYTDPLGVLICGGSTEGPGNALDNCVSIYPEADEPEWQIERMPSFRVMTCMAPLPDGTYLIANGALHGVAGFGLGVGPNLNALLYDPSKPLGSRITVAANTTIARMYHSEAITLLDGRVLISGSNPEDGVNPEEYRVEVFLPPYLLAGKPRPTFTLENRDWAHGQTGIPFTLGSPARNGDITATLLGSVASTHGNSMGARTLMPRVSCRGTSCTVDAPPTANICPPGWYQFFVLDGGIPAVGVYVRIGGDAGQIGNWPQAPDFSVPGV

SEQ ID NO: 64 - protein, 558 aa, Pycnoporus cinnabarinus; identical to SEQ ID NO: 6

SEQ ID NO: 65 - protein, 559 aa, Pycnoporus cinnabarinus
MFQTTLHLLFVLVVTGRGLAAPSTPTGWQFNLKAERSGIVALESIVVSPTLVVFFDRATNDPLQINNHSAWGALWNLETSTVRALDVLTNSFCASGALLSNGTMASIGGDPNGFPGNPAIHPGTQAIRLFEPCDSPTGEGCTLFEDPVTLHLLEKRWYPSSVRIFDGSLLIVGGMHEETPFYNTDPALSFEFFPPKESTPRPSEFLNRSLPANLFPRVFALPDGKVFMVANNQSIIYDIEANTERILPDIPNNVRVTNPIDGSAILLPLSPPDFVPEVLVCGGTQTDTIDPSLLTSQTPASSQCSRIRLDEEGIARGWEVEHMLEGRMMPELVHLPNGQVLIANGARTGFAAIASVSDPVGGSNADHAVLVPSLYTPDAPLGTRISNVGLPSSGIARVYHSSITLTPQGNFLIAGSNPNNNSSVTAGVKFPSEFRVQTLDPPFMFVERPKILSMPKKLAFGKSFTVPIAVPSTLAHPGAKVQVSLMDLGFSSHAFHSSARLVFMNAKISQDGKSLTFTTPPNGRVYPPGPATIFLTIDDVTSEGAWVMMGSGNPPPTLE

SEQ ID NO: 66 - protein, 656 aa, synthetic construct
MGHHHHHHSSGHIEGRHMASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDFEDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVT